(19) United States
(12) Patent Application Publication
(10) Pub. No.: US 2026/0200980 A1
(43) Pub. Date: Jul. 16, 2026

(54) METHODS AND COMPOSITIONS FOR TREATING OCULAR LESIONS

(71) Applicant: ONL Therapeutics, Inc., Ann Arbor, MI (US)

(72) Inventors: Andrew J. KOCAB, Farmington Hills, MI (US); David N. ZACKS, Ann Arbor, MI (US); Constance I. CHANG, Ann Arbor, MI (US); Lindsay M. PUSCAS, Northville, MI (US); Stephanie C. WIETHOLTER, Snellville, GA (US); Lori Lynn HUANG, Saline, MI (US); David A. ESPOSITO, Ann Arbor, MI (US); Sushanta MALLICK, Richardson, TX (US); Mitchell G. BRIGELL, Belmont, MA (US); David M. KLEINMAN, Rochester, NY (US); Jana VAN DE GOOR, Jerome, MI (US)

(21) Appl. No.: 19/136,727

(22) PCT Filed: Dec. 7, 2023
(86) PCT No.: PCT/US2023/082943

§ 371 Date: Jun. 6, 2025

Related U.S. Application Data
(60) Provisional application No. 63/386,856, filed on Dec. 9, 2022.
(60) Provisional application No. 63/386,859, filed on Dec. 9, 2022.
(60) Provisional application No. 63/485,214, filed on Feb. 15, 2023.
(60) Provisional application No. 63/602,101, filed on Nov. 22, 2023.

(51) Int. Cl. C07K 7/06; A61K 38/00; A61P 27/02

(52) U.S. Cl. CPC C07K 7/06; A61P 27/02; A61K 38/00

(57) ABSTRACT

Provided herein are methods and compositions for improving visual function in an eye having an ocular lesion. Provided herein are methods of treating ocular lesions in an eye of an individual, the method comprising: (a) administering a first composition comprising Fas inhibitor. In some embodiments, the method further comprises (b) administering a second composition comprising the Fas inhibitor, wherein the second composition is administered about 10 weeks or more after administering the first composition.

### CROSS REFERENCE

This application claims the benefit of U.S. Provisional Application No. 63/386,856 filed on Dec. 9, 2022, U.S. Provisional Application No. 63/386,859 filed on Dec. 9, 2022, U.S. Provisional Application No. 63/485,214 filed on Feb. 15, 2023, U.S. Provisional Application No. 63/602,101 filed on Nov. 22, 2023, each of which is incorporated by reference herein in its entirety.

### BACKGROUND

Vision is generally dependent on maintaining the anatomical and histological integrity of the structures within the eye. Changes in anatomical and histological homeostasis can provide the basis for a decrease and/or loss in vision. Ocular lesions constitute abnormal changes in the structure of the eye (e.g., retina) that can contribute to a decrease and/or loss in vision. Lesions in the retina generally arise due to injury or disease.

### SUMMARY

Provided herein are methods of improving visual function in an eye of an individual having a patchy ocular lesion, wherein the method comprises: administering a peptide (e.g., a composition comprising the peptide) to the eye, wherein the peptide comprises an amino acid sequence HHIYLGAVNYIY or variant sequence thereof, or a pharmaceutically acceptable salt thereof. Further provided are methods of improving visual function in an eye of an individual having a patchy ocular lesion, wherein the method comprises:
(a) administering a first composition to the eye, wherein the first composition comprises: a peptide comprising an amino acid sequence HHIYLGAVNYIY or variant sequence thereof, or a pharmaceutically acceptable salt thereof; and
(b) administering a second composition to the eye, wherein the second composition comprises the peptide, and wherein the second composition is administered about 10 weeks or greater after administering the first composition.
   Also provided are methods of improving visual function in an eye of an individual having a patchy ocular lesion, wherein the method comprises: administering a peptide (e.g., a composition comprising the peptide) having the structure of Formula III or a pharmaceutically acceptable salt thereof to the eye. Also provided are methods of improving visual function in an eye of an individual having a patchy ocular lesion, wherein the method comprises:
(a) administering a first composition to the eye, wherein the first composition comprises: a peptide having the structure of Formula III or a pharmaceutically acceptable salt thereof; and
(b) administering a second composition to the eye, wherein the second composition comprises the peptide, and wherein the second composition is administered about 10 weeks or greater after administering the first composition.
   In some embodiments, administering the Fas inhibitor and/or peptide reduces the rate of growth (e.g., an increase in total cross-sectional area) of the patchy ocular lesion. In some embodiments, administering the peptide reduces the rate of growth (e.g., an increase in cross-sectional area) of constituent foci within the patchy ocular lesion. In some embodiments, the patchy ocular lesion has a total size (e.g., cross-sectional area) of about 3 mm² or greater. In some embodiments, the patchy ocular lesion has a total size (e.g., cross-sectional area) of about 3 mm² to 20 mm². In some embodiments, the patchy ocular lesion comprises a retinal lesion.

In some embodiments, the second composition is administered about 8 weeks or more after administering the first composition. In some embodiments, the second composition is administered about 10 weeks or more after administering the first composition. In some embodiments, the second composition is administered about 12 weeks or more after administering the first composition. In some embodiments, the second composition is administered about 16 weeks or more after administering the first composition. In some embodiments, the second composition is administered about 20 weeks or more after administering the first composition. In some embodiments, the second composition is administered about 24 weeks or more after administering the first composition.

In some embodiments, a variant sequence comprises one or two conservative amino acid substitutions. In some embodiments, the variant sequence further comprises a deletion of one or two terminal amino acids In some embodiments. In some embodiments, a variant sequence comprises one conservative amino acid substitution. In some embodiments, the variant sequence further comprises a deletion of one terminal amino acid. In some embodiments, a variant sequence comprises two conservative amino acid substitutions. In some embodiments, the variant sequence further comprises a deletion of two terminal amino acids. In certain embodiments, the variant sequence inhibits, reduces, or prevents cell death of cells treated with FasL.

In some embodiments, treating the ocular lesion is reducing the rate of growth (e.g., an increase in cross-sectional area) of the ocular lesion. In some embodiments, the rate of lesion growth is reduced after administering the first composition, wherein administering the second composition maintains the reduced rate of lesion growth. In some embodiments, the rate of lesion growth is reduced after administering the second composition. In some embodiments, the rate of lesion growth is reduced after administering the first composition and is further reduced after administering the second composition. In certain embodiments, a reduction in lesion growth is compared to the rate of lesion growth in an eye that has not been treated with the peptide.

Further provided herein are methods treating an ocular lesion in an eye of an individual, comprising: administering a plurality of compositions to the eye, wherein: each composition of the plurality of compositions comprises a peptide, the peptide comprising an amino acid sequence HHIYLGAVNYIY or variant sequence thereof, or a pharmaceutically acceptable salt thereof; and no greater than five compositions are administered to the eye within a year. Also provided are methods treating an ocular lesion in an eye of an individual, comprising: administering a plurality of compositions to the eye, wherein: each composition of the plurality of compositions comprises a peptide, the peptide having the structure of Formula I or a pharmaceutically acceptable salt thereof; and no greater than five compositions are administered to the eye within a year. Additionally provided are methods treating an ocular lesion in an eye of an individual, comprising: administering a plurality of compositions to the eye, wherein: each composition of the plurality of compositions comprises a peptide, the peptide having the structure of Formula III or a pharmaceutically acceptable salt thereof; and no greater than five compositions are administered to the eye within a year. In some embodiments, a variant sequence comprises one or two conservative amino acid substitutions. In some embodiments, the variant sequence further comprises a deletion of one or two terminal amino acids In some embodiments. In some embodiments, a variant sequence comprises one conservative amino acid substitution. In some embodiments, the variant sequence further comprises a deletion of one terminal amino acid. In some embodiments, a variant sequence comprises two conservative amino acid substitutions. In some embodiments, the variant sequence further comprises a deletion of two terminal amino acids. In certain embodiments, the variant sequence inhibits, reduces, or prevents cell death of cells treated with FasL.

In some embodiments, no greater than four compositions are administered to the eye within a year. In some embodiments, no greater than three compositions are administered to the eye within a year. In some embodiments, no greater than two compositions are administered to the eye within a year. In some embodiments, the plurality of compositions comprises 2 compositions. In some embodiments, the plurality of compositions comprises 3 compositions. In some embodiments, the plurality of compositions comprises 4 compositions. In some embodiments, the plurality of compositions comprises 5 compositions.

In some embodiments, treating the ocular lesion is reducing the rate of growth (e.g., an increase in cross-sectional area) of the ocular lesion. In some embodiments, the lesion growth is reduced after administering a first composition, wherein administering subsequence compositions maintain the reduced rate of lesion growth. In some embodiments, lesion growth is reduced after administering multiple compositions (e.g., two or more). In some embodiments, lesion growth is reduced after administering a first composition and lesion growth is further reduced after administering an additional one (e.g., a second) or more (e.g., a third or fourth) composition.

In some embodiments, the ocular lesion is a retinal lesion. As used herein, an ocular lesion or retinal lesion generally refers to and encompasses abnormal changes in the structure of the eye (e.g., retina). In some embodiments, an eye having a lesion exhibits a decrease and/or loss in vision. In some embodiments, the lesion is associated with an ocular disease, ocular disorder, and/or ocular injury. In some embodiments, lesions are determined by fundus imagining. In some embodiments, lesions are characterized as areas of pallor with distinct edges (e.g., as appearing in a fundus image). In some embodiments, lesions comprises multiple areas of lesions within the eye (e.g., wherein the total area of the lesion is the sum of all lesion spots/areas).

In some embodiments, the ocular lesion (e.g., patchy lesion or non-patchy, singular lesion) has a size (e.g., a total cross-sectional area) of about 1 mm² or greater. In some embodiments, the ocular lesion has a size (e.g., a total cross-sectional area) of about 2 mm² or greater. In some embodiments, the ocular lesion has a size (e.g., a total cross-sectional area) of about 1 mm² or greater. In some embodiments, the ocular lesion has a size (e.g., a total cross-sectional area) of about 3 mm² or greater. In some embodiments, the ocular lesion has a size (e.g., a total cross-sectional area) of about 4 mm² or greater. In some embodiments, the ocular lesion has a size (e.g., a total cross-sectional area) of about 5 mm² or greater. In some embodiments, the ocular lesion has a size (e.g., a total cross-sectional area) of about 10 mm² or greater. In some embodiments, the ocular lesion has a size (e.g., a total cross-sectional area) of about 15 mm² or greater. In some embodiments, the ocular lesion has a size (e.g., a total cross-sectional area) of about 20 mm² or greater.

In some embodiments, the ocular lesion has a size (e.g., a total cross-sectional area) of about 1 mm² to about 20 mm². In some embodiments, the ocular lesion has a size (e.g., a total cross-sectional area) of about 2 mm² to about 20 mm². In some embodiments, the ocular lesion has a size (e.g., a total cross-sectional area) of about 3 mm² to about 20 mm². In some embodiments, the ocular lesion has a size (e.g., a total cross-sectional area) of about 3 mm² to about 20 mm². In some embodiments, the ocular lesion has a size (e.g., a total cross-sectional area) of about 5 mm² to about 20 mm². In some embodiments, the ocular lesion has a size (e.g., a total cross-sectional area) of about 1 mm² to about 15 mm². In some embodiments, the ocular lesion has a size (e.g., a total cross-sectional area) of about 2 mm² to about 15 mm². In some embodiments, the ocular lesion has a size (e.g., a total cross-sectional area) of about 3 mm² to about 15 mm². In some embodiments, the ocular lesion has a size (e.g., a total cross-sectional area) of about 3 mm² to about 20 mm². In some embodiments, the ocular lesion has a size (e.g., a total cross-sectional area) of about 5 mm² to about 15 mm².

In some embodiments, the method comprises administering each composition to the vitreous humor of the eye. In some embodiments, the peptide has a half-life in the vitreous humor greater than about 30 days. In some embodiments, the peptide has a half-life in the vitreous humor greater than about 90 days. In some embodiments, the peptide has a half-life in the vitreous humor greater than about 180 days. In some embodiments, the peptide has a half-life in the vitreous humor greater than about 200 days. In some embodiments, the method comprises using the vitreous humor as a depot to provide the peptide to retinal tissue in the eye.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-1B shows data demonstrating reducing lesion growth in patients receiving a Fas inhibitor. FIG. 1A shows lesion reduction data. FIG. 1B shows lesion images from data in FIG. 1A.

FIG. 2A-2B provides exemplary conversions of measures of visual acuity. FIG. 2A shows a first exemplary conversion table of measures of visual acuity. FIG. 2B shows a second exemplary conversion table of measures of visual acuity.

FIG. 3A-3B show representations of patchy lesions and single lesions. FIG. 3A shows examples of patchy lesions. FIG. 3B shows examples of single lesions.

FIG. 4 shows average change in letters read data compared to baseline in treated individuals having patchy and non-patchy lesions.

### DETAILED DESCRIPTION

Described and provided herein are compositions and methods useful for treating ocular lesions in an eye of an individual. In some embodiments, treating comprises improving visual function in an eye having the ocular lesion (e.g., an eye having a patchy lesion). In certain instances, the inhibiting and/or reducing lesion growth is achieved by utilizing a Fas inhibitor. The Fas-mediated inflammation signaling pathway is generally initiated by an interaction between the membrane-bound Fas ligand (FasL—a type II transmembrane protein of the TNF family) and Fas receptor, thereby leading to the activation of pro-inflammatory signaling (e.g., cytokine signaling, interleukin signaling, caspase action, etc.) and/or cell death signaling (e.g., apoptotic signaling, necrotic signaling, etc.) pathways.

### Fas Inhibitors

Provided herein are Fas inhibitors useful for modulating (e.g., inhibiting, preventing, and/or reducing, etc.) Fas-mediated signaling. In certain instances, the Fas inhibitors useful in treating, inhibiting, preventing, and/or reducing Fas-mediated inflammation. In certain instances, inhibiting, preventing, and/or reducing Fas-mediated inflammation allows for the treatment and/or prevention of lesion growth within the retina (e.g., photoreceptors and/or retinal pigment epithelium).

In some embodiments, the Fas inhibitors described herein encompass Met-derived peptides and/or fragments thereof. In some embodiments, the Met protein, also called c-Met or hepatocyte growth factor receptor (HGF receptor), is encoded by the Met gene (NCBI Gene ID 4233, Location: NC_000007.14 (116672196 . . . 116798386), UniProtKB—P0858). The Met protein is comprised of two major subunits: the α and β subunits, and Met and fragments of Met, including the extracellular domain of Met and its α subunit, have been shown to bind to Fas and prevent cells from undergoing apoptosis. In some embodiments, the Fas inhibitor comprises a Fas-inhibiting peptide (e.g., Met-derived peptide and/or fragment thereof). In some embodiments, the Fas inhibitors described herein comprises a Met-derived compound comprising the amino acid acids HHIYLGAVNYIY (His-His-Ile-Tyr-Leu-Gly-Ala-Val-Asn-Tyr-Ile-Tyr) (e.g., SEQ ID NOs: 1-8). In some embodiments, the peptide comprises the amino acid sequence HHIYLGAVNYIY or a variant sequence thereof.

As used herein, a peptide includes and/or refers to any of various natural or synthetic compounds containing two or more amino acids joined by a peptide bond that link the carboxyl group of one amino acid to the amino group of another. As also used herein, amino acid refers to and/or includes naturally occurring amino acids, unnatural amino acids, amino acid analogues and amino acid mimetics that function in a manner similar to a naturally occurring amino acids. Amino acids are generally referred to herein by either their name, the commonly known three letter symbols, or by the one-letter symbols recommended by the IUPAC-IUB Biochemical Nomenclature Commission.

In some embodiments, the Fas inhibitor peptides (e.g., a peptide comprising the amino acid sequence HHIYLGAVNYIY) comprises one or more naturally occurring amino acids. In some embodiments, the Fas inhibitor peptides (e.g., a peptide comprising the amino acid sequence HHIYLGAVNYIY) consists of naturally occurring amino acids. As used herein, naturally occurring amino acids include and/or refer to amino acids which are generally found in nature and are not manipulated by man. In some embodiments, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr).

In some embodiments, the Fas inhibitor comprises a variant sequence of the peptide (e.g., a peptide comprising the amino acid sequence HHIYLGAVNYIY). In some embodiments, amino acid substitutions can be made in the sequence of any of the polypeptides described herein, without necessarily decreasing or ablating its activity. Accordingly, in some embodiments, the variant sequence comprises one or more amino acid substitutions. In some embodiments, the variant sequence comprises one amino acid substitution. In some embodiments, the variant sequence comprises two amino acid substitutions. In some embodiments, the variant sequence comprises three amino acid substitutions. In some embodiments, substitutions include conservative substitutions (e.g., substitutions with amino acids of comparable chemical characteristics). In some embodiments, a non-polar amino acid can be substituted and replaced with another non-polar amino acid, wherein non-polar amino acids include alanine, leucine, isoleucine, valine, glycine, proline, phenylalanine, tryptophan and methionine. In some embodiments, a neutrally charged polar amino acids can be substituted and replaced with another neutrally charged polar amino acid, wherein neutrally charged polar amino acids include serine, threonine, cysteine, tyrosine, asparagine, and glutamine. In some embodiments, a positively charged amino acid can be substituted and replaced with another positively charged amino acid, wherein positively charged amino acids include arginine, lysine and histidine. In some embodiments, a negatively charged amino acid can be substituted and replaced with another negatively charged amino acid, wherein negatively charged amino acids include aspartic acid and glutamic acid. Examples of amino acid substitutions also include substituting an L-amino acid for its corresponding D-amino acid, substituting cysteine for homocysteine or other non-natural amino acids.

In some embodiments, the Fas inhibitor peptides (e.g., a peptide comprising the amino acid sequence HHIYLGAVNYIY) comprises one or more non-natural amino acids. In some embodiments, the Fas inhibitor peptides (e.g., a peptide comprising the amino acid sequence HHIYLGAVNYIY) consists of non-natural amino acids. As used herein, non-natural amino acids and/or unnatural amino acids include and/or refer to amino acid structures that cannot be generated biosynthetically in any organism using unmodified or modified genes from any organism. In some embodiments, non-natural amino acids and/or unnatural amino acids further include and/or refer to an amino acid residue that are not present in the naturally occurring (wild-type) Met protein sequence. For example, these include, but are not limited to, modified amino acids and/or amino acid analogues that are not one of the 20 naturally occurring amino acids (e.g., non-natural side chain variant sequence amino acids), D-amino acids, homo amino acids, beta-homo amino acids, N-methyl amino acids, alpha-methyl amino acids, or. By way of further example, non-natural amino acids also include 4-Benzoylphenylalanine (Bpa), Aminobenzoic Acid (Abz), Aminobutyric Acid (Abu), Aminohexanoic Acid (Ahx), Aminoisobutyric Acid (Aib), Citrulline (Cit), Diaminobutyric Acid (Dab), Diaminopropanoic Acid (Dap), Diaminopropionic Acid (Dap), Gamma-Carboxyglutamic Acid (Gla), Homoalanine (Hala), Homoarginine (Harg), Homoasparagine (Hasn), Homoaspartic Acid (Hasp), Homocysteine (Hcys), Homoglutamic Acid (Hglu), Homoglutamine (Hgln), Homoisoleucine (Hile), Homoleucine (Hleu), Homomethionine (Hmet), Homophenylalanine (Hphe), Homoserine (Hser), Homotyrosine (Htyr), Homovaline (Hval), Hydroxyproline (Hyp), Isonipecotic Acid (Inp), N aphthylalanine (Nal), Nipecotic Acid (Nip), Norleucine (Nle), Norvaline (Nva), Octahydroindole-2-carboxylic Acid (Oic), Penicillamine (Pen), Phenylglycine (Phg), Pyroglutamic Acid (Pyr), Sarcosine (Sar), tButylglycine (Tle), and Tetrahydro-isoquinoline-3-carboxylic Acid (Tic). Such non-natural amino acid residues can be introduced by substitution of naturally occurring amino acids, and/or by insertion of non-natural amino acids into the naturally occurring (wild-type) Met protein sequence. A non-natural amino acid residue also can be incorporated such that a desired functionality is imparted to the apelin molecule, for example, the ability to link a functional moiety (e.g., PEG).

In some embodiments, a variant sequence comprises one or more amino acid deletions. In some embodiments, the variant sequence comprises one amino acid deletion. In some embodiments, the variant sequence comprises two amino acid deletions. In some embodiments, the variant sequence comprises three amino acid deletions. In some embodiments, the variant sequence comprises four amino acid deletions. In some embodiments, the variant sequence comprises one or more additional amino acids. In some embodiments, the additional amino acids are additional amino acids from the Met sequence. In some embodiments, the variant sequence comprises a substitution and a deletion. In some embodiments, the variant sequence comprises a substitution and one or more additional amino acids. In some embodiments, the substitution comprises a natural amino acid or a non-natural amino acid. In some embodiments, the variant sequence is a retro inverso amino acid sequence. In some embodiments, a variant sequence comprises one or more additional amino acid residues (e.g., one, two, or three additions) to the N or C terminus. In some embodiments, a variant sequence comprises one or more deletions (e.g., one, two, or three deletions) to amino acid residues at the N or C terminus.

In some embodiments, a variant sequence comprises one or two conservative amino acid substitutions. In some embodiments, the variant sequence further comprises a deletion of one or two terminal amino acids In some embodiments. In some embodiments, a variant sequence comprises one conservative amino acid substitution. In some embodiments, the variant sequence further comprises a deletion of one terminal amino acid. In some embodiments, a variant sequence comprises two conservative amino acid substitutions. In some embodiments, the variant sequence further comprises a deletion of two terminal amino acids. In certain embodiments, the variant sequence inhibits, reduces, or prevents cell death of cells treated with FasL.

Functionality of variant sequences of the peptide (e.g., a variant sequence of the amino acid sequence HHIYLGAVNYIY) can be determined by an in vitro assay. For example, in some embodiments, the variant sequence competes for binding to a Fas receptor (FasR) with Fas ligand (FasL). In some embodiments, the variant sequence inhibits, reduces, or prevents caspase 8 activation in cells treated with FasL (e.g., as measured by commercially available luminescent tetrapeptide cleavage assay kit (Promega, Madison, WI)). In some embodiments, the variant sequence inhibits, reduces, or prevents cell death of cells treated with FasL. By way of further example, in some embodiments, the variant sequence competes for binding to a Fas receptor (FasR) with a Fas-activating antibody (e.g., Fas-agonistic Jo2 monoclonal antibody (BD Biosciences, San Jose, CA)). In some embodiments, the variant sequence inhibits, reduces, or prevents caspase 8 activation in cells treated with a Fas-activating antibody (e.g., as measured by commercially available luminescent tetrapeptide cleavage assay kit (Promega, Madison, WI)). In some embodiments, the variant sequence inhibits, reduces, or prevents cell death of cells treated with a Fas-activating antibody. Accordingly, in some embodiments, the Fas inhibitor comprises a variant sequence (e.g., any one of the variant sequences described herein) of the peptide (e.g., a peptide comprising the amino acid sequence HHIYLGAVNYIY), wherein the variant sequence competes for binding to a Fas receptor (FasR) with Fas ligand (FasL). In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence inhibits, reduces, or prevents caspase 8 activation in cells treated with FasL. In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence inhibits, reduces, or prevents cell death of cells treated with FasL. In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence competes for binding to a Fas receptor (FasR) with a Fas-activating antibody. In some embodiments, the Fas inhibitor comprises a variant sequence (e.g., any one of the variant sequences described herein) of the peptide comprising the amino acid sequence HHIYLGAVNYIY, wherein the variant sequence inhibits, reduces, or prevents caspase 8 activation in cells treated with a Fas-activating antibody. In some embodiments, the Fas inhibitor comprises a variant sequence (e.g., any one of the variant sequences described herein) of the peptide comprising the amino acid sequence HHIYLGAVNYIY, wherein the variant sequence inhibits, reduces, or prevents cell death of cells treated with a Fas-activating antibody.

The peptide or a variant sequence thereof can further comprise one or more modifications. In some embodiments, the peptide (e.g., a comprising the amino acid sequence HHIYLGAVNYIY or a variant sequence thereof) comprises a modification. In some embodiments, the peptide is a modified peptide. As used herein, a modification or a modified peptide includes and/or refers to a modification of one or more amino acids in the peptide. In some embodiments, modifications species of stereoisomers. All stereoisomers of the above compounds are contemplated, either in admixture or in pure or substantially pure form. The compounds can have asymmetric centers at any of the atoms. Consequently, the peptide compounds or components thereof can exist in enantiomeric or diastereomeric forms or in mixtures thereof. The present invention contemplates the use of any racemates (i.e., mixtures containing equal amounts of each enantiomer), enantiomerically enriched mixtures (i.e., mixtures enriched for one enantiomer), pure enantiomers or diastereomers, or any mixtures thereof. The chiral centers can be designated as R or S or R, S or d, D, 1, L or d, 1, D, or L. Compounds comprising amino acid residues include residues of D-amino acids, L-amino acids, or racemic derivatives of amino acids. Compounds comprising sugar residues include residues of D-sugars, L-sugars, or racemic derivatives of sugars. Non-limiting examples of modifications are phosphorylation, glycosylation, ubiquitination, nitrosylation, methylation, acetylation, amidation, or lipidation. Modification can be introduced at the C-terminus of the peptide, the N-terminus of the peptide, or at any place in-between. Thus, a modification or a modified peptide includes and/or refers to modifications of the free amino- and/or carboxyl-terminal (N-terminus and C-terminus, respectively). In some embodiments, N-terminal modifications include but are not limited to acetylation, formylation, pyroglutamylation, carbamide addition, lipidation, sulfonamidation, and alkylamination. In some embodiments, C-terminal modifications include but are not limited to amidation, esterification, and incorporation of an aldehyde group. In some embodiments, the modification comprises amidation. In some embodiments, the amidation is at the c-terminus. In some embodiments, the modification comprises a retro inverso peptide (e.g., YIYNVAGLYIHH). In some embodiments, the modification altering the chirality of one or more amino acid residues of the peptide (e.g., L amino acid to D amino acid).

Accordingly, in an embodiment, provided herein are peptides comprising the sequence (a)-HHIYLGAVNYIY-(b) (SEQ ID NO: 1) or (a)-YIYNVAGLYIHH-(b) (SEQ ID NO: 2), or a variant sequence thereof, wherein:
(a) is —H, —OH, —NH₂, G¹(CH₂)ₙ—, R¹CONH—, or R²O—;
(b) is —H, —CH₂OH, —CH₂OR², —CHO, —CO₂R², —CONH₂, —CONHR², —CON(R³)₂, —CONH(CH₂)_{y}NR(³)₂, —(CH₂)ₙ-G¹, —COCH₂-G¹, —CONHCH₂-G¹, —(CH₂)ₙNH₂, —(CH₂)ₙNHR², —(CH₂)ₙN(R³)₂, NH-Glu-His-OH, NH-Glu-His-NH₂, -Ala-His-NH₂, -Gly-His-NH₂, —NH-Glu-His-OH, —NH-Glu-His-NH₂, -Ala-His-NH₂, -Gly-His-NH₂, —NH-[D]Glu-[D]-His-OH, —NH-[D]Glu-[D]-His-NH₂, -[D]Ala-[D]-His-NH₂, -Gly[D]-His-NH₂, or —CONH(CH₂)ₙ-G²; G¹, at each occurrence, is independently —H, —C(═O)NH₂, —C(═O)NHR², —C(═O)N(R³)₂, C(═O)OR², or —C(═O)R¹; G² at each occurrence is a heterocyclic ring of 4-7 members comprising at least one tertiary amine functionality NR² within the ring, or a carbocyclic ring of 3-7 members substituted with —N(R³)₂;
R¹, at each occurrence, is independently H, C₁₋₆alkyl, —(CH₂)ₓ(OCH₂CH₂)ₘOR⁵, C₁₋₆-alkoxy or L;
R², at each occurrence, is independently C₁₋₆alkyl, C₂₋₆alkyl substituted with OR⁵ or NR⁵₂, —(CH₂)ₓ(OCH₂CH₂)ₘOR⁵ or L;
L, at each occurrence, is a multivalent polyethylene glycol derivative with 2-4 termini, each of which may be independently capped with H, R⁵;
R³, at each occurrence, is independently C₁₋₆alkyl, C₂₋₆alkyl substituted with OR⁵ or N(R⁵)₂, —(CH₂)ₓ(OCH₂CH₂)ₘOR⁵;
or two R³s, taken together with the N atom to which they are attached, may form a monocyclic ring of 4-8 members or a fused, bridged or spiro bicyclic ring of 6-10 members, which can include up to two groups within the ring chosen independently from —O—, —(C═O)—, NR⁶, S, SO, or SO₂;
R⁴, at each occurrence, is independently C₁₋₆alkyl, C₁₋₆acyl, or —OPO₃(R⁵)₂;
R⁵, at each occurrence, is independently H or C₁₋₆alkyl;
R⁶, at each occurrence, is H, C₁₋₆alkyl, C₂₋₆hydroxyalkyl, C₁₋₆alkoxy-, C₁₋₆alkyl, or C₁₋₆acyl;
m=1-100;
n=0-3;
x=0-6; and
y=2-4,
wherein at most one of R¹ and R² is L.

In certain instances, provided herein are peptides comprising the structure of Formula I or Formula II, or a pharmaceutically acceptable salt thereof.

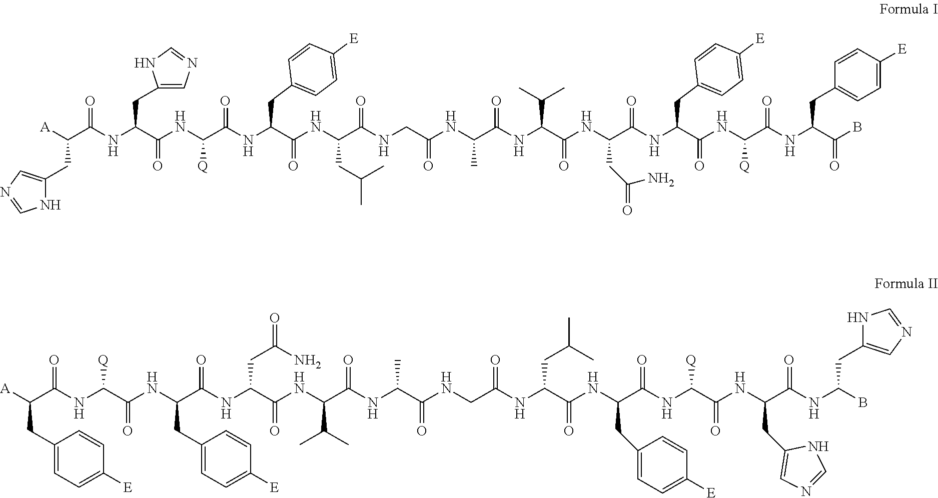

wherein:
A is H—, —OH, —NH₂, G(CH₂)ₙ—, R¹CONH—, or R²O—;
B is —H, CH₂OH, CH₂OR², —CHO, —CO₂R², —CONH₂, —CONHR², —CON(R³)₂, —CONH(CH₂)_{y}N(R³)₂, —(CH₂)ₙ-G¹, —COCH₂-G¹, —CONHCH₂-G¹, —(CH₂)ₙNH₂, —(CH₂)ₙNHR², —(CH₂)ₙN(R³)₂ NH-Glu-His-OH, NH-Glu-His-NH₂, -Ala-His-NH₂, -Gly-His-NH₂, NH-Glu-His-OH, NH-Glu-His-NH₂, -Ala-His-NH₂, -Gly-His-NH₂, NH-[D]Glu-[D]-His-OH, NH-[D]Glu-[D]-His-NH₂, -[D]Ala-[D]-His-NH₂, -Gly-[D]-His-NH₂, or CONH(CH₂)ₙ-G²;
E, at each occurrence, is independently —H, —OH, —OR⁴, SH, —SR⁴, or halogen;
G¹, at each occurrence, is independently —H, —C(═O)NH₂, —C(═O)NHR², —C(═O)N(R³)₂, C(═O)OR², or —C(═O)R¹;
G² at each occurrence is a heteroalicyclic ring of 4-7 members comprising at least one tertiary amine functionality NR² within the ring, or a carbocyclic ring of 3-7 members substituted with N(R³)₂;
Q, at each occurrence, is independently, 1-propyl, 2-propyl, 2-methyl-prop-2-yl, C₃₋₆-cycloalkyl, C₄₋₆-cycloalkenyl, tetrahydrofuran-2-yl, tetrahydrofuran-3-yl, tetrahydrothienyl-2-yl, tetrahydrothienyl-3-yl, tetrahydropyran-2-yl, tetrahydropyran-3-yl, tetrahydropyran-4-yltetrahydrothiopyran-2-yl, tetrahydrothiopyran-3-yl, tetrahydrothiopyran-4-yl or 1-CH(OR⁵)CH₃;
R¹, at each occurrence, is independently H, C₁₋₆alkyl, —(CH₂)ₓ(OCH₂CH₂)ₘOR⁵, C₁₋₆alkoxy or L;
R², at each occurrence, is independently C₁₋₆alkyl, C₂₋₆alkyl substituted with OR⁵ or N(R⁵)₂, —(CH₂)ₓ(OCH₂CH₂)ₘOR⁵ or L;
L, at each occurrence, is a multivalent polyethylene glycol derivative with 2-4 termini, each of which may be independently capped with H, R⁵ or another molecule of the peptide of Formula I or II;
R³, at each occurrence, is independently C₁₋₆-alkyl, C₂₋₆-alkyl substituted with OR⁵ or N(R⁵)₂, —(CH₂)ₓ(OCH₂CH₂)ₘOR⁵;
or two R³s, taken together with the N atom to which they are attached, may form a monocyclic ring of 4-8 members or a fused, bridged or spiro bicyclic ring of 6-10 members, which can include up to two groups within the ring chosen independently from —O—, —(C═O)—, NR⁶, S, SO, or SO₂;
R⁴, at each occurrence, is independently C₁₋₆alkyl, C₁₋₆acyl, or —OPO₃(R⁵)₂;
R⁵, at each occurrence, is independently H or C₁₋₆alkyl;
R⁶, at each occurrence, is H, C₁₋₆alkyl, C₂₋₆hydroxyalkyl, C₁₋₆alkoxy-, C₁₋₆alkyl, or C₁₋₆acyl;
m=1-100;
n=0-3;
x=0-6; and
y=2-4,
wherein at most one of R¹ and R² is L.

In some embodiments, provided is a peptide having the structure of Formula III or a pharmaceutically acceptable salt thereof:

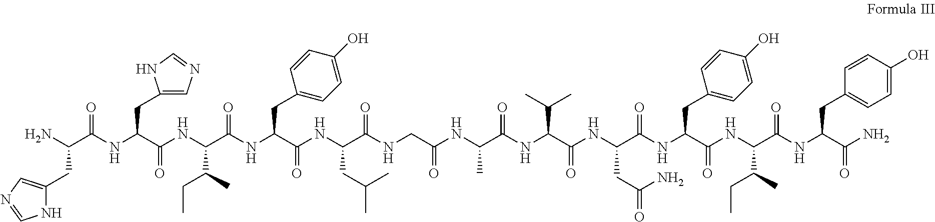 [SMILES: CC[C@H](C)[C@H](NC(=O)[C@H](Cc1ccc(O)cc1)NC(=O)[C@H](CC(N)=O)NC(=O)[C@@H](NC(=O)[C@H](C)NC(=O)CNC(=O)[C@H](CC(C)C)NC(=O)[C@H](Cc1ccc(O)cc1)NC(=O)[C@@H](NC(=O)[C@H](Cc1cnc[nH]1)NC(=O)[C@@H](N)Cc1cnc[nH]1)[C@@H](C)CC)C(C)C)C(=O)N[C@@H](Cc1ccc(O)cc1)C(N)=O]

In some embodiments, provided is a peptide having the structure of Formula IV or a pharmaceutically acceptable salt thereof:

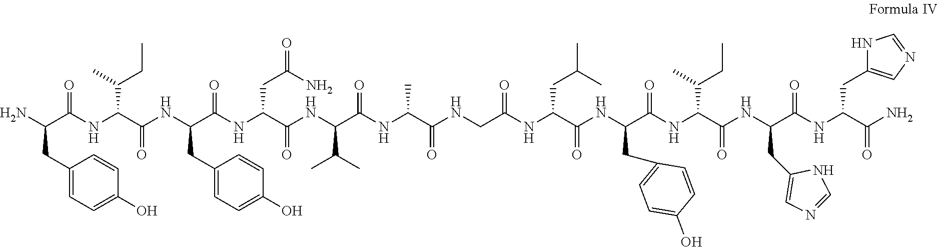 [SMILES: CC[C@@H](C)[C@@H](NC(=O)[C@H](N)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@H](C)CC)C(C)C]

In some embodiments, provided is a peptide having the structure of Formula V or a pharmaceutically acceptable salt thereof:

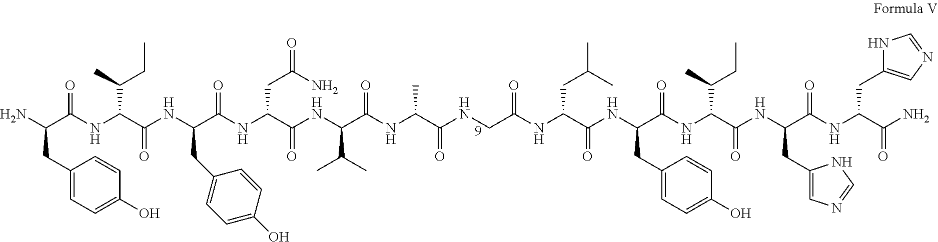 [SMILES: CC[C@H](C)[C@@H](NC(=O)[C@H](N)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@@H](C)CC)C(C)C]

In some embodiments, provided is a peptide having the structure of Formula VI or a pharmaceutically acceptable salt thereof:

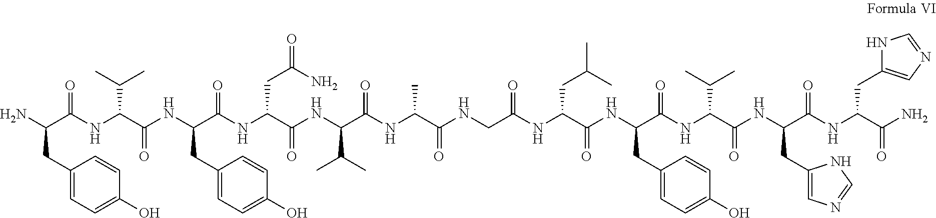 [SMILES: CC(C)C[C@@H](NC(=O)CNC(=O)[C@@H](C)NC(=O)[C@H](NC(=O)[C@@H](CC(N)=O)NC(=O)[C@@H](Cc1ccc(O)cc1)NC(=O)[C@H](NC(=O)[C@H](N)Cc1ccc(O)cc1)C(C)C)C(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)C(C)C]

In some embodiments, provided is a peptide having the structure of Formula VII or a pharmaceutically acceptable salt thereof:

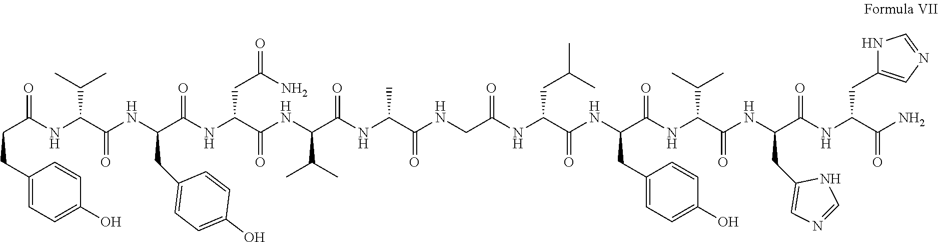

In some embodiments, provided is a peptide having the structure of Formula VIII or a pharmaceutically acceptable salt thereof:

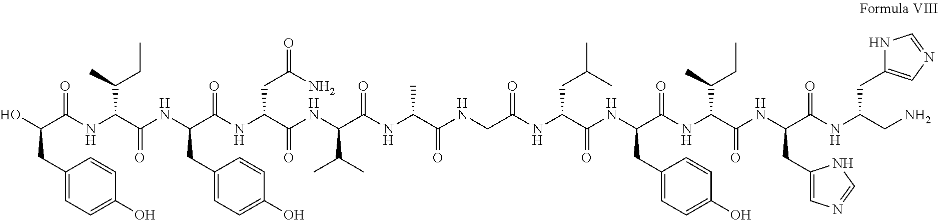 [SMILES: CC[C@H](C)[C@@H](NC(=O)[C@H](O)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@@H](CN)Cc1cnc[nH]1)[C@@H](C)CC)C(C)C]

In some embodiments, provided is a peptide having the structure of Formula IX or a pharmaceutically acceptable salt thereof:

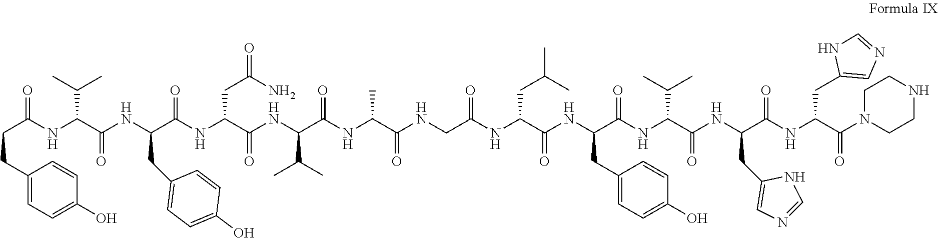

### Salts of Fas Inhibiting Peptides

Further provided herein are salts of the peptide for inhibiting Fas-mediated inflammation in the eye and for use in the methods described here. As used herein, salt is generally synonymous with pharmaceutically acceptable salts, and/or includes or refers to pharmaceutically acceptable salts. Examples of pharmaceutically acceptable salts are salts with organic or inorganic acids such as (but not limited to) include acetic acid, aspartic acid, benzenesulfonic acid, benzoic acid, butyric acid, citric acid, fumaric acid, hydrochloric acid, hydrobromic acid, lactic acid, maleic acid, malonic acid, methanesulfonic acid, 4-methylbenzenesulfonic acid, nicotinic acid, phosphoric acid, succinic acid, sulfuric acid, or tartaric acid, prepared using methods well known in the art. In some embodiments, the salt is a hydrochloride salt.

In addition, these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, and polyamine resins.

Salts and pharmaceutically acceptable salts are described in in J. Pharmaceutical Sciences, 66: 1-19 (1977), the contents of which are incorporated by reference herein.

In some embodiments, the salt is an acetate salt. In some embodiments, the acetate salt is a poly-acetate salt. In some embodiments, the poly-acetate salt is a tri-acetate salt.

### Pharmaceutical Compositions

In an embodiment, further provided are pharmaceutical compositions (also referred to as compositions) comprising the Fas inhibiting peptides. In some embodiments, the pharmaceutical compositions described herein comprise the peptide (e.g., a peptide comprising the amino acid sequence HHIYLGAVNYIY) or a pharmaceutically acceptable salt thereof. In some embodiments, the pharmaceutical compositions described herein comprise the peptide comprising the amino acid sequence HHIYLGAVNYIY or variant sequence thereof, or a pharmaceutically acceptable salt of the peptide. In some embodiments, the pharmaceutical compositions described herein comprise the peptide having the structure of any one of Formulas I-IX or a pharmaceutically acceptable salt thereof. In some embodiments, the pharmaceutical compositions described herein comprise the peptide having the structure of Formula I or a pharmaceutically acceptable salt thereof. In some embodiments, the pharmaceutical compositions described herein comprise the peptide having the structure of Formula III or a pharmaceutically acceptable salt thereof.

The pharmaceutical composition can comprise one or more excipients. As used herein, an excipient includes and/or refers to any pharmaceutically acceptable additive, carrier, diluent, adjuvant, or other ingredient, other than the active pharmaceutical ingredient (API), which is typically included for formulation and/or administration to a patient. A pharmaceutical composition can comprise a single pharmaceutical formulation (e.g., extended release, immediate release, delayed release, nanoparticulate, etc.) or multiple formulations (e.g., immediate release and delayed release, nanoparticulate and nonnanoparticulate, etc.). An excipient further includes and/or refers to an agent that may be added to a formulation to provide a desired consistency (e.g., altering the bulk properties), to improve stability, and/or to adjust osmolality. Examples of commonly used excipients include, but are not limited to, sugars, polyols, amino acids, surfactants, and polymers. In some embodiments, a non-ionic excipient or a non-ionizable excipient, as used herein, includes and/or refers to an agent having no net charge.

In some embodiments, the non-ionic excipient has no net charge under certain formulation conditions, such as pH. Examples of non-ionic excipients include, but are not limited to, sugars (e.g., sucrose), sugar alcohols (e.g., mannitol), and non-ionic surfactants (e.g., polysorbate 80).

In some embodiments, the compositions comprise excipients that are suitable for ocular application. Suitable excipients and include, but are not limited to, tonicity agents, preservatives, chelating agents, buffering agents, surfactants, cosolvents and antioxidants. Suitable tonicity-adjusting agents include mannitol, sodium chloride, glycerin, sorbitol and the like. Suitable preservatives include p-hydroxybenzoic acid ester, benzalkonium chloride, benzododecinium bromide, polyquaternium-1, and the like. Suitable chelating agents include sodium edetate and the like. Suitable buffering agents include phosphates, borates, citrates, acetates, tromethamine, and the like. Suitable surfactants include ionic and nonionic surfactants. In some embodiments, the one or more excipients comprises nonionic surfactants, such as polysorbates, polyethoxylated castor oil derivatives, polyethoxylated fatty acids, polyethoxylated alcohols, polyoxyethylene-polyoxypropylene block copolymers (Poloxamer), and oxyethylated tertiary octylphenol formaldehyde polymer (Tyloxapol). Other suitable surfactants may also be included. Suitable antioxidants include sulfites, thiosulfate, ascorbates, BHA, BHT, tocopherols, and the like.

In some embodiments, the composition comprises a non-ionic surfactant. In some embodiments, the composition comprises a polysorbate, a polyethoxylated castor oil derivative, a polyethoxylated fatty acid, a polyethoxylated alcohol, a polyoxyethylene-polyoxypropylene block copolymer (Poloxamer), or an oxyethylated tertiary octylphenol formaldehyde polymer (Tyloxapol). In some embodiments, the composition comprises a polysorbate. In some embodiments, the composition comprises a polyethoxylated castor oil derivative. In some embodiments, the composition comprises a polyethoxylated fatty acid. In some embodiments, the composition comprises a polyethoxylated alcohol. In some embodiments, the composition comprises a polyoxyethylene-polyoxypropylene block copolymer (Poloxamer). In some embodiments, the composition comprises an oxyethylated tertiary octylphenol formaldehyde polymer (Tyloxapol). In some embodiments, the surfactant makes up 0.05%-20% weight per weight (w/w) of the composition. In some embodiments, the non-ionic surfactant is about 0.05% w/w of the composition to about 20% w/w of the composition. In some embodiments, the non-ionic surfactant is at least about 0.05% w/w of the composition. In some embodiments, the non-ionic surfactant is at most about 20% w/w of the composition. In some embodiments, the non-ionic surfactant is about 0.05% w/w of the composition to about 0.1% w/w of the composition, about 0.05% w/w of the composition to about 0.5% w/w of the composition, about 0.05% w/w of the composition to about 1% w/w of the composition, about 0.05% w/w of the composition to about 2% w/w of the composition, about 0.05% w/w of the composition to about 5% w/w of the composition, about 0.05% w/w of the composition to about 10% w/w of the composition, about 0.05% w/w of the composition to about 20% w/w of the composition, about 0.1% w/w of the composition to about 0.5% w/w of the composition, about 0.1% w/w of the composition to about 1% w/w of the composition, about 0.1% w/w of the composition to about 2% w/w of the composition, about 0.1% w/w of the composition to about 5% w/w of the composition, about 0.1% w/w of the composition to about 10% w/w of the composition, about 0.1% w/w of the composition to about 20% w/w of the composition, about 0.5% w/w of the composition to about 1% w/w of the composition, about 0.5% w/w of the composition to about 2% w/w of the composition, about 0.5% w/w of the composition to about 5% w/w of the composition, about 0.5% w/w of the composition to about 10% w/w of the composition, about 0.5% w/w of the composition to about 20% w/w of the composition, about 1% w/w of the composition to about 2% w/w of the composition, about 1% w/w of the composition to about 5% w/w of the composition, about 1% w/w of the composition to about 10% w/w of the composition, about 1% w/w of the composition to about 20% w/w of the composition, about 2% w/w of the composition to about 5% w/w of the composition, about 2% w/w of the composition to about 10% w/w of the composition, about 2% w/w of the composition to about 20% w/w of the composition, about 5% w/w of the composition to about 10% w/w of the composition, about 5% w/w of the composition to about 20% w/w of the composition, or about 10% w/w of the composition to about 20% w/w of the composition. In some embodiments, the non-ionic surfactant is about 0.05% w/w of the composition, about 0.1% w/w of the composition, about 0.5% w/w of the composition, about 1% w/w of the composition, about 2% w/w of the composition, about 5% w/w of the composition, about 10% w/w of the composition, or about 20% w/w of the composition.

In some embodiments, the non-ionic surfactant is about 0.05% w/w of the composition to about 2% w/w of the composition. In some embodiments, the non-ionic surfactant is at least about 0.05% w/w of the composition. In some embodiments, the non-ionic surfactant is at most about 2% w/w of the composition. In some embodiments, the non-ionic surfactant is about 0.05% w/w of the composition to about 0.1% w/w of the composition, about 0.05% w/w of the composition to about 0.1% w/w of the composition, about 0.05% w/w of the composition to about 0.2% w/w of the composition, about 0.05% w/w of the composition to about 0.3% w/w of the composition, about 0.05% w/w of the composition to about 0.4% w/w of the composition, about 0.05% w/w of the composition to about 0.5% w/w of the composition, about 0.05% w/w of the composition to about 0.6% w/w of the composition, about 0.05% w/w of the composition to about 1% w/w of the composition, about 0.05% w/w of the composition to about 1.5% w/w of the composition, about 0.05% w/w of the composition to about 2% w/w of the composition, about 0.1% w/w of the composition to about 0.1% w/w of the composition, about 0.1% w/w of the composition to about 0.2% w/w of the composition, about 0.1% w/w of the composition to about 0.3% w/w of the composition, about 0.1% w/w of the composition to about 0.4% w/w of the composition, about 0.1% w/w of the composition to about 0.5% w/w of the composition, about 0.1% w/w of the composition to about 0.6% w/w of the composition, about 0.1% w/w of the composition to about 1% w/w of the composition, about 0.1% w/w of the composition to about 1.5% w/w of the composition, about 0.1% w/w of the composition to about 2% w/w of the composition, about 0.1% w/w of the composition to about 0.2% w/w of the composition, about 0.1% w/w of the composition to about 0.3% w/w of the composition, about 0.1% w/w of the composition to about 0.4% w/w of the composition, about 0.1% w/w of the composition to about 0.5% w/w of the composition, about 0.1% w/w of the composition to about 0.6% w/w of the composition, about 0.1% w/w of the composition to about 1% w/w of the composition, about 0.1% w/w of the composition to about 1.5% w/w of the composition, about 0.1% w/w of the composition to about 2% w/w of the composition, about 0.2% w/w of the composition to about 0.3% w/w of the composition, about 0.2% w/w of the composition to about 0.4% w/w of the composition, about 0.2% w/w of the composition to about 0.5% w/w of the composition, about 0.2% w/w of the composition to about 0.6% w/w of the composition, about 0.2% w/w of the composition to about 1% w/w of the composition, about 0.2% w/w of the composition to about 1.5% w/w of the composition, about 0.2% w/w of the composition to about 2% w/w of the composition, about 0.3% w/w of the composition to about 0.4% w/w of the composition, about 0.3% w/w of the composition to about 0.5% w/w of the composition, about 0.3% w/w of the composition to about 0.6% w/w of the composition, about 0.3% w/w of the composition to about 1% w/w of the composition, about 0.3% w/w of the composition to about 1.5% w/w of the composition, about 0.3% w/w of the composition to about 2% w/w of the composition, about 0.4% w/w of the composition to about 0.5% w/w of the composition, about 0.4% w/w of the composition to about 0.6% w/w of the composition, about 0.4% w/w of the composition to about 1% w/w of the composition, about 0.4% w/w of the composition to about 1.5% w/w of the composition, about 0.4% w/w of the composition to about 2% w/w of the composition, about 0.5% w/w of the composition to about 0.6% w/w of the composition, about 0.5% w/w of the composition to about 1% w/w of the composition, about 0.5% w/w of the composition to about 1.5% w/w of the composition, about 0.5% w/w of the composition to about 2% w/w of the composition, about 0.6% w/w of the composition to about 1% w/w of the composition, about 0.6% w/w of the composition to about 1.5% w/w of the composition, about 0.6% w/w of the composition to about 2% w/w of the composition, about 1% w/w of the composition to about 1.5% w/w of the composition, about 1% w/w of the composition to about 2% w/w of the composition, or about 1.5% w/w of the composition to about 2% w/w of the composition. In some embodiments, the non-ionic surfactant is about 0.05% w/w of the composition, about 0.1% w/w of the composition, about 0.1% w/w of the composition, about 0.2% w/w of the composition, about 0.3% w/w of the composition, about 0.4% w/w of the composition, about 0.5% w/w of the composition, about 0.6% w/w of the composition, about 1% w/w of the composition, about 1.5% w/w of the composition, or about 2% w/w of the composition.

In some embodiments, the non-ionic surfactant comprises Polysorbate 20, Poloxamer 407, Tyloxapol, or cremophor. In some embodiments, the non-ionic surfactant is Polysorbate 20. In some embodiments, the non-ionic surfactant is Poloxamer 407. In some embodiments, the non-ionic surfactant is Tyloxapol. In some embodiments, the non-ionic surfactant is cremophor. The non-ionic surfactants described herein can be present within any one of the ranges (e.g., percent w/w) described herein, a specific value that falls within the described ranges.

In some embodiments, the composition further comprises cosolvents (e.g., between 0.5 and 50% w/w), such as N,N-Dimethylacetamide, ethanol, PEG-400, propylene glycol, dimethylsulfoxide (DMSO); oils, or cyclodextrins may be added to a pharmaceutical preparation. In some embodiments, the composition further comprises a tonicity-adjusting agent. In some embodiments, the composition is an isotonic solution. In some embodiments, the tonicity-adjusting agent is mannitol, sorbitol, glucose or trehalose, or an inorganic salt such as sodium chloride. In some embodiments, the composition comprises mannitol. In some embodiments, the composition comprises sorbitol. In some embodiments, the composition comprises glucose or trehalose. In some embodiments, the composition comprises an inorganic salt. In some embodiments, the tonicity-adjusting agent is present at an amount suitable to bring the tonicity of the composition into the 250-400 mOsm/L range. In some embodiments, the non-ionic surfactant is about 1% w/w of the composition to about 10% w/w of the composition. In some embodiments, the non-ionic surfactant is at least about 1% w/w of the composition. In some embodiments, the non-ionic surfactant is at most about 10% w/w of the composition. In some embodiments, the non-ionic surfactant is about 1% w/w of the composition to about 2% w/w of the composition, about 1% w/w of the composition to about 3% w/w of the composition, about 1% w/w of the composition to about 4% w/w of the composition, about 1% w/w of the composition to about 5% w/w of the composition, about 1% w/w of the composition to about 10% w/w of the composition, about 2% w/w of the composition to about 3% w/w of the composition, about 2% w/w of the composition to about 4% w/w of the composition, about 2% w/w of the composition to about 5% w/w of the composition, about 2% w/w of the composition to about 10% w/w of the composition, about 3% w/w of the composition to about 4% w/w of the composition, about 3% w/w of the composition to about 5% w/w of the composition, about 3% w/w of the composition to about 10% w/w of the composition, about 4% w/w of the composition to about 5% w/w of the composition, about 4% w/w of the composition to about 10% w/w of the composition, or about 5% w/w of the composition to about 10% w/w of the composition. In some embodiments, the non-ionic surfactant is about 1% w/w of the composition, about 2% w/w of the composition, about 3% w/w of the composition, about 4% w/w of the composition, about 5% w/w of the composition, or about 10% w/w of the composition.

In some embodiments, the composition comprises a buffering agent. In some embodiments, the buffering agent is an acidifying agent. In some embodiments, the acidifying agent is an acetate buffer at pH 4.5. In some embodiments, the concentration acetate buffer pH 4.5 is about 10 millimolar (mM). Generally, the pH may be controlled by an appropriate buffer suitable for injection into the eye, for example the pH of the composition can be in the 3.0-7.5 range or 3.5-4.5 range.

As described herein, the composition can comprise one or more excipients. Accordingly, in some embodiments, the composition comprises a non-ionic surfactant, a tonicity-adjusting agent, and a buffering agent, in combination with the peptide. Any of the described excipients can be combined within the amounts and/or ranges described.

### Dosages and Dosing Regimens

In an embodiment, the compositions described herein comprise an amount of the peptide suitable to inhibit Fas-mediated inflammation and/or treat, inhibit and/or reduce ocular lesion growth or the symptoms thereof in an eye. As used herein, a dose or dosage includes and/or refers to the amount of therapeutic agent, such as the peptides described, in a composition (e.g., a composition for administering to an eye). A dose can refer to either (i) the peptide (parent compound) or the pharmaceutically acceptable salt thereof. In some embodiments, the amount of the peptide in the composition (i.e., pharmaceutical composition) that is suitable for the methods described herein (e.g., treating retinal lesions) ranges from 5 micrograms (ug) to 10,000 ug. The dosing forms comprising the compositions described herein are generally administered to the vitreous humor of an eye and can be further formulated for injection into the eye (e.g., intravitreal injection). In some embodiments, the amount of the peptide (e.g., a peptide comprising the amino acid sequence HHIYLGAVNYIY) or a pharmaceutically acceptable salt thereof that is suitable for the methods described herein ranges from 5 ug to 10,000 ug. In some embodiments, the amount of the peptide comprising the amino acid sequence HHIYLGAVNYIY or variant sequence thereof, or a pharmaceutically acceptable salt of the peptide that is suitable for the methods described herein ranges from 5 ug to 10,000 ug. In some embodiments, the amount of the peptide having the structure of Formula I or a pharmaceutically acceptable salt thereof that is suitable for the methods described herein ranges from 5 ug to 10,000 ug. In some embodiments, the amount of the peptide having the structure of Formula III or a pharmaceutically acceptable salt thereof that is suitable for the methods described herein ranges from 5 ug to 10,000 ug.

In some embodiments, a dose comprises about 5-1,000 ug of the peptide (e.g., Formula III) or the variant sequence thereof. In some embodiments, a dose comprises about 25-500 ug of the peptide or the variant sequence thereof. In some embodiments, a dose comprises about 25-250 ug of the peptide or the variant sequence thereof. In some embodiments, a dose comprises about 50-250 ug of the peptide or the variant sequence thereof. In certain embodiments, a dose comprises about 50 ug of the peptide or the variant sequence thereof. In certain embodiments, a dose comprises about 100 ug of the peptide or the variant sequence thereof. In certain embodiments, a dose comprises about 200 ug of the peptide or the variant sequence thereof. In certain embodiments, a dose comprises about 300 ug of the peptide or the variant sequence thereof. In some embodiments, the peptide is present at a concentration 0.1 milligrams per milliliter (mg/mL) to 10 mg/mL. In some embodiments, the peptide is present at a concentration 0.1 milligrams per milliliter (mg/mL) to 5.0 mg/mL.

In some embodiments, a dose comprises about 5 ug of a pharmaceutically acceptable salt of the peptide to about 300 ug of a pharmaceutically acceptable salt of the peptide. In some embodiments, a dose comprises at least about 5 ug of a pharmaceutically acceptable salt of the peptide. In some embodiments, a dose comprises at most about 300 ug of a pharmaceutically acceptable salt of the peptide.

The concentration of the peptide within the composition can be adjusted in a manner suitable for ocular administration. In some embodiments, the concentration of the peptide within the composition ranges from about 0.1 milligrams per milliliter (mg/mL) to about 5 mg/mL. In some embodiments, the concentration of the peptide within the composition ranges from about 0.1 milligrams per milliliter (mg/mL) to about 10 mg/mL. In some embodiments, the concentration of the peptide within the composition ranges from about 0.1 milligrams per milliliter (mg/mL) to about 100 mg/mL. In some embodiments, the concentration of the peptide (e.g., a peptide comprising the amino acid sequence HHIYLGAVNYIY) or a pharmaceutically acceptable salt thereof ranges from about 0.1 mg/mL to about 5 mg/mL. In some embodiments, the concentration of the peptide comprising the amino acid sequence HHIYLGAVNYIY or variant sequence thereof, or a pharmaceutically acceptable salt of the peptide ranges from about 0.1 milligrams per milliliter (mg/mL) to about 5 mg/mL. In some embodiments, the concentration of the peptide having the structure of Formula I or a pharmaceutically acceptable salt thereof ranges from about 0.1 mg/mL to about 5 mg/mL. In some embodiments, the concentration of the peptide having the structure of Formula III or a pharmaceutically acceptable salt thereof ranges from about 0.1 mg/mL to about 5 mg/mL.

### Pharmacokinetics

In some embodiments, the compositions described herein are administered to an eye of an individual in need thereof. Administration to an eye (i.e., “ocular application” or “ocular administration”) includes subconjunctival, intravitreal, retrobulbar, intracameral administration subretinal, or suprachoroidal. In some embodiments, ocular administration comprises subconjunctival, intravitreal, retrobulbar, or intracameral administration. In some embodiments, ocular administration comprises intravitreal administration. In some embodiments, ocular administration comprises subconjunctival administration. In some embodiments, ocular administration comprises retrobulbar administration. In some embodiments, ocular administration comprises intracameral administration.

In some embodiments, the dosing forms comprising the compositions described herein are generally administered to the vitreous humor of an eye. In some embodiments, the half-life of the peptide (e.g., a peptide comprising the amino acid sequence HHIYLGAVNYIY) or a pharmaceutically acceptable salt thereof in the vitreous humor is greater than about 30 days to greater than about 275 days. In some embodiments, the peptide comprising the amino acid sequence HHIYLGAVNYIY or variant sequence thereof, or a pharmaceutically acceptable salt of the peptide has a half-life in the vitreous humor that is greater than about 30 days to greater than about 275 days. In some embodiments, the peptide having the structure of Formula I or a pharmaceutically acceptable salt thereof has a half-life in the vitreous humor that is greater than is greater than about 30 days to greater than about 275 days. In some embodiments, the peptide having the structure of Formula III or a pharmaceutically acceptable salt thereof has a half-life in the vitreous humor that is greater than is greater than about 30 days to greater than about 275 days. In some embodiments, the peptide comprising the amino acid sequence HHIYLGAVNYIY or variant sequence thereof, or a pharmaceutically acceptable salt of the peptide has a half-life in the vitreous humor that is greater than about 14 days to greater than about 275 days. In some embodiments, the peptide having the structure of Formula I or a pharmaceutically acceptable salt thereof has a half-life in the vitreous humor that is greater than is greater than about 14 days to greater than about 275 days. In some embodiments, the peptide having the structure of Formula III or a pharmaceutically acceptable salt thereof has a half-life in the vitreous humor that is greater than is greater than about 14 days to greater than about 275 days.

In some embodiments, the half-life of the peptide is greater than about 14 days in the eye. In some embodiments, the half-life of the peptide is greater than about 30 days in the eye. In some embodiments, the half-life of the peptide is greater than about 60 days in the eye. In some embodiments, the half-life of the peptide is greater than about 90 days in the eye. In some embodiments, the half-life of the peptide is greater than about 120 days in the eye. In some embodiments, the half-life of the peptide is greater than about 150 days in the eye. In some embodiments, the half-life of the peptide is greater than about 180 days in the eye. In some embodiments, the half-life of the peptide is greater than about 210 days in the eye. In some embodiments, the half-life of the peptide is greater than about 240 days in the eye. In some embodiments, the half-life of the peptide is greater than about 270 days in the eye.

In some embodiments, the half-life of the peptide is greater than about 14 days in the vitreous humor. In some embodiments, the half-life of the peptide is greater than about 30 days in the vitreous humor. In some embodiments, the half-life of the peptide is greater than about 60 days in the vitreous humor. In some embodiments, the half-life of the peptide is greater than about 90 days in the vitreous humor. In some embodiments, the half-life of the peptide is greater than about 120 days in the vitreous humor. In some embodiments, the half-life of the peptide is greater than about 150 days in the vitreous humor. In some embodiments, the half-life of the peptide is greater than about 180 days in the vitreous humor. In some embodiments, the half-life of the peptide is greater than about 210 days in the vitreous humor. In some embodiments, the half-life of the peptide is greater than about 240 days in the vitreous humor. In some embodiments, the half-life of the peptide is greater than about 270 days in the vitreous humor.

Determining the amount of the peptide in the vitreous humor generally requires collecting all of the vitreous fluid or a substantial portion thereof from an eye or sacrificing the eye in order to sample the vitreous humor. In some embodiments, collecting all of the vitreous fluid or a substantial portion thereof in a human eye, or sacrificing an eye is not feasible for maintaining the health of an eye in a human. Accordingly, in some embodiments, the half-life of the peptide in a human eye is determined by measuring and/or extrapolating from a half-life of the peptide in the eye of a mammal. In some embodiments, the mammal is a rabbit. In some embodiments, the mammal is a pig (e.g., minipig). In some embodiments, the mammal is a monkey. Various methods of detecting the presence of a drug are also suitable for detecting the peptide. For example, methods suitable for detecting the peptide include performing mass spectrometry (e.g., liquid chromatography-mass spectrometry (LC-MS) or high-performance LC-MS (HPLC-MS)) on a sample from the vitreous humor.

### Methods

Provided herein are methods that are advantageous for use in treating ocular lesions. In some embodiments, an eye comprising an ocular lesion comprises a loss in visual function (e.g., best corrected visual acuity, field of vision, contrast sensitivity, binocular function, low luminance acuity, low contrast acuity, color vision, perimetry, threshold sensitivity, reading speed, and/or light/dark adaptation). In certain embodiments, treating the ocular lesion further treats the loss in visual function. In some embodiments, provided are methods of treating a loss in visual function in an eye having an ocular lesion. In some embodiments, provided are methods of improving visual function in an eye having an ocular lesion.

Provided herein are methods are methods of treating an ocular lesion in an eye of an individual, comprising: (a) administering a Fas inhibitor to the eye of the individual. In some embodiments, the Fas inhibitor is a peptide, a polypeptide (e.g., FasR or FasL), an antibody that binds FasR, or antibody that binds FasL.

Provided herein are methods are methods of treating an ocular lesion in an eye of an individual, comprising: (a) administering a peptide to the eye of the individual, the peptide comprising an amino acid sequence HHIYLGAVNYIY or a variant sequence thereof, or a pharmaceutically acceptable salt of the peptide. Further provided are methods of treating an ocular lesion in an eye of an individual having an ocular disease or disorder, comprising: (a) administering a peptide to the eye of the individual, the peptide having the structure of Formula I or a pharmaceutically acceptable salt thereof. Also provided are methods of treating an ocular lesion in an eye of an individual having an ocular disease or disorder, comprising: (a) administering a peptide to the eye of the individual, the peptide having the structure of Formula III or a pharmaceutically acceptable salt thereof. In some embodiments, treating the ocular lesion is reducing the rate of growth (e.g., an increase in cross-sectional area) of the ocular lesion.

In some embodiments, further provided herein are methods of neuroprotection within an eye of an individual, comprising: administering a Fas inhibitor to the eye. In certain instances, neuroprotection comprises reducing the rate of lesion growth, inhibiting lesion growth, and/or protecting against increased lesion growth. In some embodiments, provided herein are methods of neuroprotection within an eye of an individual (e.g., reducing the rate of lesion growth, inhibiting lesion growth, and/or protecting against increased lesion growth), comprising: administering a peptide to the eye of the individual, wherein the peptide comprises an amino acid sequence HHIYLGAVNYIY or a variant sequence thereof, or a pharmaceutically acceptable salt of the peptide. In some embodiments, provided herein are methods of neuroprotection within an eye of an individual (e.g., reducing the rate of lesion growth, inhibiting lesion growth, and/or protecting against increased lesion growth), comprising: administering a peptide to the eye of the individual, the peptide having the structure of Formula I or a pharmaceutically acceptable salt thereof. In some embodiments, provided herein are methods of neuroprotection within an eye of an individual (e.g., reducing the rate of lesion growth, inhibiting lesion growth, and/or protecting against increased lesion growth), comprising: administering a peptide to the eye, the peptide having the structure of Formula III or a pharmaceutically acceptable salt thereof. In certain embodiments, neuroprotection comprises reducing the rate of lesion growth, inhibiting lesion growth, and/or protecting against increased lesion growth (e.g., relative to a control or untreated eye having a lesion).

Further provided herein are methods of treating an ocular lesion in an eye of an individual, comprising: (a) administering a first composition comprising a peptide to the eye of the individual, the peptide comprising an amino acid sequence HHIYLGAVNYIY or a variant sequence thereof, or a pharmaceutically acceptable salt of the peptide; and (b) administering a second composition comprising the peptide to the eye, wherein the second composition is administered about 10 weeks or more after administering the first composition. Also provided are methods of treating an ocular lesion in an eye of an individual, comprising: (a) administering a first composition comprising a peptide to the eye of the individual, the peptide having the structure of Formula I or a pharmaceutically acceptable salt thereof; and (b) administering a second composition comprising the peptide to the eye, wherein the second composition is administered about 10 weeks or more after administering the first composition. Additionally provided are methods of treating an ocular lesion in an eye of an individual, comprising: (a) administering a first composition comprising a peptide to the eye of the individual, the peptide having the structure of Formula III or a pharmaceutically acceptable salt thereof; and (b) administering a second composition comprising the peptide to the eye, wherein the second composition is administered about 10 weeks or more after administering the first composition.

In some embodiments, provided are methods of improving visual function in an eye of an individual having a patchy ocular lesion, wherein the method comprises: administering a peptide (e.g., a composition comprising the peptide) to the eye, wherein the peptide comprises an amino acid sequence HHIYLGAVNYIY or variant sequence thereof, or a pharmaceutically acceptable salt thereof.

Further provided are methods of improving visual function in an eye of an individual having a patchy ocular lesion, wherein the method comprises:
(a) administering a first composition to the eye, wherein the first composition comprises: a peptide comprising an amino acid sequence HHIYLGAVNYIY or variant sequence thereof, or a pharmaceutically acceptable salt thereof; and
(b) administering a second composition to the eye, wherein the second composition comprises the peptide, and wherein the second composition is administered about 10 weeks or greater after administering the first composition.

Also provided are methods of improving visual function in an eye of an individual having a patchy ocular lesion, wherein the method comprises: administering a peptide (e.g., a composition comprising the peptide) having the structure of Formula I or a pharmaceutically acceptable salt thereof to the eye. Also provided are methods of improving visual function in an eye of an individual having a patchy ocular lesion, wherein the method comprises:
(a) administering a first composition to the eye, wherein the first composition comprises: a peptide having the structure of Formula I or a pharmaceutically acceptable salt thereof; and
(b) administering a second composition to the eye, wherein the second composition comprises the peptide, and wherein the second composition is administered about 10 weeks or greater after administering the first composition.

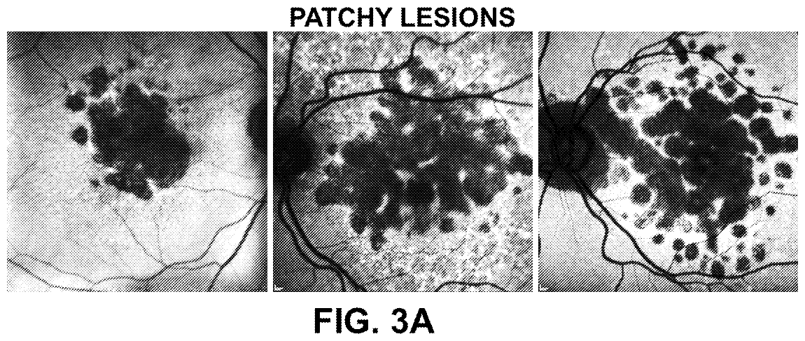

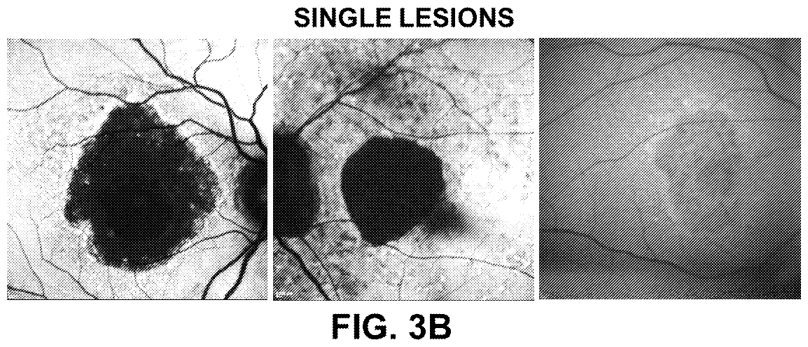

Further provided are methods of improving visual function in an eye of an individual having a patchy ocular lesion, wherein the method comprises: administering a peptide (e.g., a composition comprising the peptide) having the structure of Formula III or a pharmaceutically acceptable salt thereof to the eye. Also provided are methods of improving visual function in an eye of an individual having a patchy ocular lesion, wherein the method comprises:
(a) administering a first composition to the eye, wherein the first composition comprises: a peptide having the structure of Formula III or a pharmaceutically acceptable salt thereof; and
(b) administering a second composition to the eye, wherein the second composition comprises the peptide, and wherein the second composition is administered about 10 weeks or greater after administering the first composition.
   Examples of patchy lesions are shown in FIG. 3A, as compared to single lesions (e.g., non-patchy lesions) shown in FIG. 3B. “Patchy lesions” can also be referred to as “multifocal lesions” or “speckled lesions” or “non-homogeneous hypo-autofluorescence” lesions (e.g., as opposed to homogeneous and/or dense hypo-autofluorescence). In certain embodiments, patchy ocular lesions refer to and encompass an eye having multiple (e.g., two or more) smaller lesions. In certain instances, the patchy ocular lesion size can be determined by the summed size (e.g., total surface area) of the multiple smaller lesions. In some embodiments, the total area of the patchy ocular lesion comprises the summed area of multiple smaller lesions. In some embodiments, the methods comprise identifying and/or detecting (e.g., by autofluorescence) the patchy lesion in the eye prior to administering the peptide. In some embodiments, administering the peptide reduces the rate of growth (e.g., an increase in total cross-sectional area) of the patchy ocular lesion. In some embodiments, administering the peptide reduces the rate of growth (e.g., an increase in cross-sectional area) of constituent foci within the patchy ocular lesion. In some embodiments, the patchy ocular lesion has a total size (e.g., cross-sectional area) of about 3 mm² or greater. In some embodiments, the patchy ocular lesion has a total size (e.g., cross-sectional area) of about 3 mm² to 20 mm². In some embodiments, the patchy ocular lesion comprises a retinal lesion.

In some embodiments, the second composition is administered about 8 weeks or more after administering the first composition. In some embodiments, the second composition is administered about 10 weeks or more after administering the first composition. In some embodiments, the second composition is administered about 12 weeks or more after administering the first composition. In some embodiments, the second composition is administered about 16 weeks or more after administering the first composition. In some embodiments, the second composition is administered about 20 weeks or more after administering the first composition. In some embodiments, the second composition is administered about 24 weeks or more after administering the first composition.

In some embodiments, treating the ocular lesion is reducing the rate of growth (e.g., an increase in cross-sectional area) of the ocular lesion. In some embodiments, the rate of lesion growth is reduced after administering the first composition, wherein administering the second composition maintains the reduced rate of lesion growth. In some embodiments, the rate of lesion growth is reduced after administering the second composition. In some embodiments, the rate of lesion growth is reduced after administering the first composition and is further reduced after administering the second composition. In certain embodiments, a reduction in lesion growth is compared to the rate of lesion growth in an eye that has not been treated with the peptide.

Further provided herein are methods treating an ocular lesion in an eye of an individual, comprising: administering a plurality of compositions to the eye, wherein: each composition of the plurality of compositions comprises a peptide, the peptide comprising an amino acid sequence HHIYLGAVNYIY or variant sequence thereof, or a pharmaceutically acceptable salt thereof; and no greater than five compositions are administered to the eye within a year. Also provided are methods treating an ocular lesion in an eye of an individual, comprising: administering a plurality of compositions to the eye, wherein: each composition of the plurality of compositions comprises a peptide, the peptide having the structure of Formula I or a pharmaceutically acceptable salt thereof; and no greater than five compositions are administered to the eye within a year. Additionally provided are methods treating an ocular lesion in an eye of an individual, comprising: administering a plurality of compositions to the eye, wherein: each composition of the plurality of compositions comprises a peptide, the peptide having the structure of Formula III or a pharmaceutically acceptable salt thereof; and no greater than five compositions are administered to the eye within a year.

In some embodiments, no greater than four compositions are administered to the eye within a year. In some embodiments, no greater than three compositions are administered to the eye within a year. In some embodiments, no greater than two compositions are administered to the eye within a year. In some embodiments, the plurality of compositions comprises 2 compositions. In some embodiments, the plurality of compositions comprises 3 compositions. In some embodiments, the plurality of compositions comprises 4 compositions. In some embodiments, the plurality of compositions comprises 5 compositions.

In some embodiments, treating the ocular lesion is reducing the rate of growth (e.g., an increase in cross-sectional area) of the ocular lesion. In some embodiments, the lesion growth is reduced after administering a first composition, wherein administering subsequence compositions maintain the reduced rate of lesion growth. In some embodiments, lesion growth is reduced after administering multiple compositions (e.g., two or more). In some embodiments, lesion growth is reduced after administering a first composition and lesion growth is further reduced after administering an additional one (e.g., a second) or more (e.g., a third or fourth) composition.

In some embodiments, the ocular lesion is a retinal lesion. As used herein, an ocular lesion or retinal lesion generally refers to and encompasses abnormal changes in the structure of the eye (e.g., retina). In some embodiments, an eye having a lesion exhibits a decrease and/or loss in vision. In some embodiments, the lesion is associated with an ocular disease, ocular disorder, and/or ocular injury. In some embodiments, lesions are determined by fundus imagining. In some embodiments, lesions are characterized as areas of pallor with distinct edges (e.g., as appearing in a fundus image). In some embodiments, lesions comprises multiple areas of lesions within the eye (e.g., wherein the total area of the lesion is the sum of all lesion spots/areas).

In some embodiments, the ocular lesion has a size (e.g., a total cross-sectional area) of about 1 mm² or greater. In some embodiments, the ocular lesion has a size (e.g., a total cross-sectional area) of about 2 mm² or greater. In some embodiments, the ocular lesion has a size (e.g., a total cross-sectional area) of about 1 mm² or greater. In some embodiments, the ocular lesion has a size (e.g., a total cross-sectional area) of about 3 mm² or greater. In some embodiments, the ocular lesion has a size (e.g., a total cross-sectional area) of about 4 mm² or greater. In some embodiments, the ocular lesion has a size (e.g., a total cross-sectional area) of about 5 mm² or greater. In some embodiments, the ocular lesion has a size (e.g., a total cross-sectional area) of about 10 mm² or greater. In some embodiments, the ocular lesion has a size (e.g., a total cross-sectional area) of about 15 mm² or greater. In some embodiments, the ocular lesion has a size (e.g., a total cross-sectional area) of about 20 mm² or greater.

In some embodiments, the ocular lesion has a size (e.g., a total cross-sectional area) of about 1 mm² to about 20 mm². In some embodiments, the ocular lesion has a size (e.g., a total cross-sectional area) of about 2 mm² to about 20 mm². In some embodiments, the ocular lesion has a size (e.g., a total cross-sectional area) of about 3 mm² to about 20 mm 2. In some embodiments, the ocular lesion has a size (e.g., a total cross-sectional area) of about 3 mm² to about 20 mm². In some embodiments, the ocular lesion has a size (e.g., a total cross-sectional area) of about 5 mm² to about 20 mm². In some embodiments, the ocular lesion has a size (e.g., a total cross-sectional area) of about 1 mm² to about 15 mm². In some embodiments, the ocular lesion has a size (e.g., a total cross-sectional area) of about 2 mm² to about 15 mm². In some embodiments, the ocular lesion has a size (e.g., a total cross-sectional area) of about 3 mm² to about 15 mm². In some embodiments, the ocular lesion has a size (e.g., a total cross-sectional area) of about 3 mm² to about 20 mm². In some embodiments, the ocular lesion has a size (e.g., a total cross-sectional area) of about 5 mm² to about 15 mm².

In certain embodiments, the ocular lesion further comprises areas and/or regions of cell atrophy and/or cell death. In certain embodiments, the ocular lesion further comprises areas and/or regions of retinal cell atrophy and/or retinal cell death.

In some embodiments, the method comprises administering each composition to the vitreous humor of the eye. In some embodiments, the peptide has a half-life in the vitreous humor greater than about 30 days. In some embodiments, the peptide has a half-life in the vitreous humor greater than about 90 days. In some embodiments, the peptide has a half-life in the vitreous humor greater than about 180 days. In some embodiments, the peptide has a half-life in the vitreous humor greater than about 200 days. In some embodiments, the method comprises using the vitreous humor as a depot to provide the peptide to retinal tissue in the eye. In certain embodiments, the peptide is present in the vitreous humor greater than about 8 weeks after administration. In certain embodiments, the peptide is present in the vitreous humor greater than about 10 weeks after administration. In certain embodiments, the peptide is present in the vitreous humor greater than about 12 weeks after administration. In certain embodiments, the peptide is present in the vitreous humor greater than about 16 weeks after administration. In certain embodiments, the peptide is present in the vitreous humor greater than about 20 weeks after administration. In certain embodiments, the peptide is present in the vitreous humor greater than about 24 weeks after administration. In certain embodiments, the peptide is present in the vitreous humor greater than about 30 weeks after administration. In certain embodiments, the peptide is present in the vitreous humor greater than about 40 weeks after administration. In certain embodiments, the peptide is present in the vitreous humor greater than about 50 weeks after administration.

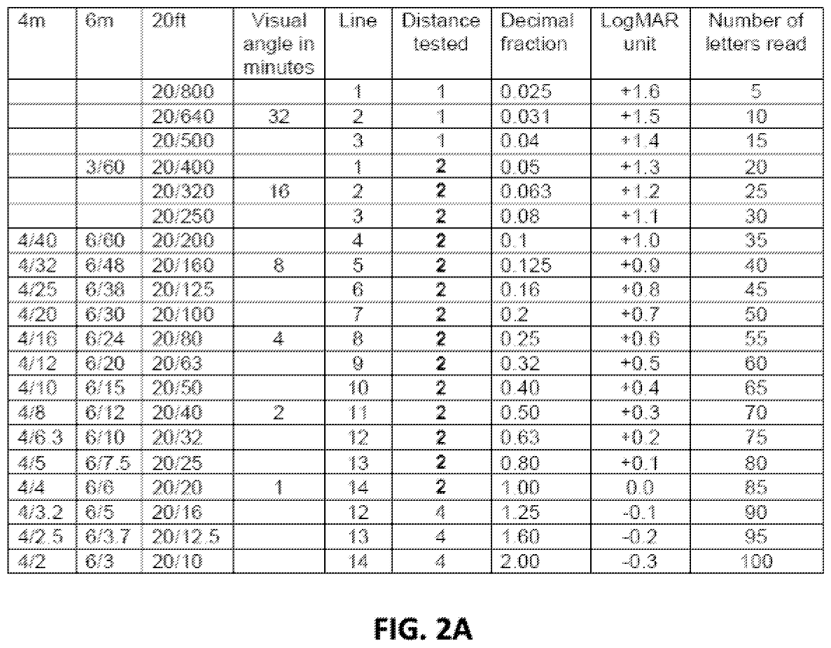

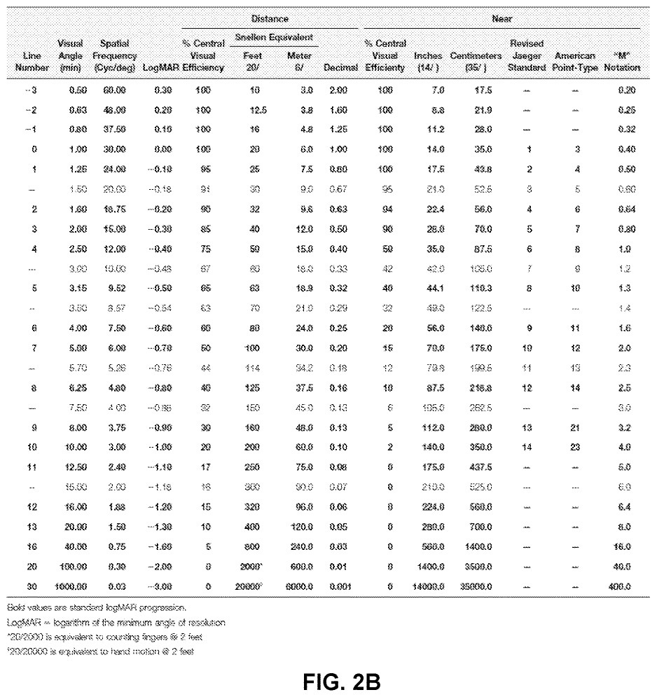

In some embodiments, treating a loss visual function in an eye having a lesion comprises reducing a loss (e.g., rate of loss) in visual function as compared to a baseline visual function prior to administering the peptide or the variant sequence thereof, or the pharmaceutically acceptable salt of the peptide. In some embodiments, treating a loss visual function in an eye having a lesion comprises increasing and/or improving visual function (e.g., improving BCVA) as compared to a baseline visual function prior to administering the peptide or the variant sequence thereof, or the pharmaceutically acceptable salt of the peptide. In some embodiments, the methods comprise increasing and/or improving visual function (e.g., improving BCVA) as compared to a baseline visual function prior to administering the peptide or the variant sequence thereof, or the pharmaceutically acceptable salt of the peptide. In some embodiments, visual function comprises one or more measurements selected from the group consisting of: best corrected visual acuity (e.g., letters read), field of vision, contrast sensitivity, binocular function, low luminance acuity, low contrast acuity, color vision, perimetry, threshold sensitivity, reading speed, and light/dark adaptation. In some embodiments, improving visual function comprises improving one or more measurements selected from the group consisting of: best corrected visual acuity (e.g., letters read), field of vision, contrast sensitivity, binocular function, low luminance acuity, low contrast acuity, color vision, perimetry, threshold sensitivity, reading speed, and light/dark adaptation. In some embodiments, visual function comprises best corrected visual acuity (e.g., increase in visual acuity from 20/200 to 20/100, or an increase in visual acuity from 20/50 to 20/40, and/or an increase in visual acuity from measured in a reduction in logMAR, etc.). As used herein, the term “best corrected visual acuity” or “BCVA”, generally refers to the minimum angle of resolution subtended by a certain number of arc minutes. In certain instances, the subtended visual angle of an object is the angle formed by rays projecting from the eye to the top and bottom (or left and right sides) of an object. In such instances, such visual angles are used to indicate the size of the retinal image of the object (e.g., the larger the visual angle, the larger the retinal image size is). In certain instances, the visual angle is influenced by two parameters: the size of the object and the distance of the object from the eye. Bigger objects cast larger images on the retina than smaller objects. Thus, the larger the object is, the larger its visual angle will be. Closer objects cast larger images on the retina than smaller objects. Thus, the closer the object is to the eye, the larger its visual angle will be. By way of example, standard vision is thus the ability to distinguish features separated by 1 minute of arc. By way of further example, the ability to distinguish a set of bars separated by 1 arc minute is 20/20 vision, 2 arc minutes is 20/40 vision. In some embodiments, visual acuity is measured by a Snellen chart, ETDRS chart, Landolt C chart, Tumbling E chart, HOTV chart, and/or logMAR (log minimum angle of resolution). FIG. 2A-2B show an exemplary, but non-limiting, conversion chart for comparing BCVA values. In some embodiments, visual function comprises field of vision. In some embodiments, visual function comprises contrast sensitivity. In some embodiments, visual function comprises binocular function. In some embodiments, visual function comprises low luminance acuity. In some embodiments, visual function comprises low contrast acuity. In some embodiments, visual function comprises color vision. In some embodiments, visual function comprises perimetry. In some embodiments, visual function comprises threshold sensitivity. In some embodiment, visual function comprises reading speed. In some embodiments, visual function comprises light/dark adaptation. In some embodiments, treating visual function comprises reducing a loss in visual function as compared to a baseline visual function prior to the administering peptide or the variant sequence thereof, or the pharmaceutically acceptable salt of the peptide.

In some embodiments, the methods described herein include methods of treating vision loss associated with inflammation (e.g., Fas-mediated inflammation) in an eye. In some embodiments, retinal inflammation can be determined by observing the symptoms associated with inflammation in the eye (e.g., loss of and/or decrease in visual acuity, central vision loss, blurred vision, distorted vision, etc.) and/or by a biological assay detecting the presence of inflammatory molecules (e.g., inflammatory cytokines) in a sample (e.g., vitreous humor sample) taken from the eye. Exemplary inflammatory molecules include, but are not limited to, Fas-mediated inflammation-related molecules (e.g. TNFa, IL-1b, IP-10, IL-18, MIP-1a, IL-6, GFAP, MIP2, MCP-1, or MIP-1b); a Fas-mediated complement-related molecules (complement component 3 (C3) or complement component 1 q (C1q)) Caspase 8; components of the inflammasome (e.g., NLRP3 or NLRP2); C-X-C motif chemokines (e.g., CXCL2 (MIP-2alpha) or CXCL10 (IP-10)); C-X3-C motif chemokines (e.g., CX3CL1 (fractalkine)); C-C motif chemokines (CCL2 (MCP-1), CCL3 (MIP-1a), and CCL4 (MIP-1b)); toll-like receptor 4 (TLR4); interleukin cytokines (e.g., IL-1b, IL-18, and IL-6); TNF superfamily cytokines (e.g., TNFa); or GFAP.

In some embodiments, the variant sequence comprises an amino acid substitution. In some embodiments, the variant sequence comprises one amino acid substitution. In some embodiments, the variant sequence comprises two amino acid substitutions. In some embodiments, the variant sequence comprises three amino acid substitutions. In some embodiments, the variant sequence comprises a truncation.

In some embodiments, the peptide further comprises a modification. In some embodiments, comprises a modified amino acid or a non-natural amino acid. In some embodiments, the peptide comprises an amidated C-terminus. In some embodiments, the peptide has the structure of Formula I, or a pharmaceutically acceptable salt thereof. In some embodiments, the peptide has the structure of Formula III, or a pharmaceutically acceptable salt thereof.

In some embodiments, the pharmaceutically acceptable salt of the peptide is administered. In some embodiments, the pharmaceutically acceptable salt is an acetate salt. In some embodiments, the pharmaceutically acceptable salt is a polyacetate salt. In some embodiments, the polyacetate salt is a triacetate salt. In some embodiments, the pharmaceutically acceptable salt is a hydrochloride salt.

In some embodiments, the peptide or the variant sequence thereof, or the pharmaceutically acceptable salt of the peptide is formulated in a composition (e.g., the pharmaceutical compositions described herein).

In some embodiments, about 5-1,000 ug of the peptide or the variant sequence thereof, or the pharmaceutically acceptable salt of the peptide is administered. In some embodiments, about 25-500 ug of the peptide or the variant sequence thereof, or the pharmaceutically acceptable salt of the peptide is administered. In some embodiments, about 25-250 ug of the peptide or the variant sequence thereof, or the pharmaceutically acceptable salt of the peptide is administered. In some embodiments, about 50-250 ug of the peptide or the variant sequence thereof, or the pharmaceutically acceptable salt of the peptide is administered. In certain embodiments, about 50 ug of the peptide or the variant sequence thereof, or the pharmaceutically acceptable salt of the peptide is administered. In certain embodiments, about 100 ug of the peptide or the variant sequence thereof, or the pharmaceutically acceptable salt of the peptide is administered. In certain embodiments, about 200 ug of the peptide or the variant sequence thereof, or the pharmaceutically acceptable salt of the peptide is administered. In some embodiments, the peptide is present at a concentration 0.1 milligrams per milliliter (mg/mL) to 10 mg/mL. In some embodiments, the peptide is present at a concentration 0.1 milligrams per milliliter (mg/mL) to 5.0 mg/mL.

As used herein, individual is synonymous with patient and/or subject and includes and/or refers to a human and may be a human that has been diagnosed as needing to treat a disease or condition as disclosed herein. However, examples are not limited to humans and include, chimpanzees, marmosets, cows, horses, sheep, goats, pigs, rabbits, dogs, cats, rats, mice, guinea pigs, and the like. The individual is typically a human and may be a human that has been diagnosed as needing to treat a disease or condition as disclosed herein.

As used herein, the term “inhibition” or “inhibiting” or reducing includes and/or refers to the reduction or suppression of a given condition, symptom, disorder, or disease, and/or a decrease in the baseline activity of a biological activity or process.

As used herein, the term “treating” or “treatment” of includes and/or refers to ameliorating the disease or disorder or symptoms thereof (e.g., slowing or arresting or reducing the development of the disease or at least one of the clinical symptoms thereof). In certain embodiments, “treating” or “treatment” also includes and/or refers to alleviating or ameliorating at least one physical and/or biological parameters including those which may not be discernible by the patient. In certain embodiments, “treating” or “treatment” includes and/or refers to modulating a disease, disorder, or biological process either physically, (e.g., stabilization of a discernible symptom), physiologically, (e.g., stabilization of a physical and/or biological parameter), or both. In certain embodiments, “treating” or “treatment” includes and/or refers to preventing or delaying the onset or development or progression of the disease or disorder. In certain embodiments, “treating” or “treatment” includes and/or refers to preventing or delaying or inhibiting the deterioration of (i) a healthy physiological state or (ii) a baseline physiological state (e.g., the progression of a disease or disorder).

As used herein, “a” or “an” when used in conjunction with the term “comprising” in the claims and/or the specification includes and/or refers to “one” and also consistent with the meaning of “one or more”, “at least one”, and “one or more than one”. Similarly, the word “another” may mean at least a second or more.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used herein, the term “about” in the context of a given value or range includes and/or refers to a value or range that is within 20%, within 10%, and/or within 5% of the given value or range.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

As used herein, a “sample” includes and/or refers to any fluid or liquid sample which is being analyzed in order to detect and/or quantify an analyte. In some embodiments, a sample is a biological sample. Examples of samples include without limitation a bodily fluid, an extract, a solution containing proteins and/or DNA, a cell extract, a cell lysate, or a tissue lysate. Non-limiting examples of bodily fluids include urine, saliva, blood, serum, plasma, cerebrospinal fluid, tears, semen, sweat, pleural effusion, liquified fecal matter, and lacrimal gland secretion.

As used herein, in any instance or embodiment described herein, “comprising” may be replaced with “consisting essentially of” and/or “consisting of”, used herein, in any instance or embodiment described herein, “comprises” may be replaced with “consists essentially of” and/or “consists of”.

### EXAMPLES

### Example I—Reducing Ocular Lesion Growth Patients

### Objective

The primary objective was to determine safety of a peptide having the structure of Formula III (ONL1204) and select doses in patients having an ocular lesion. The primary endpoints were the safety profile associated with single and multiple dosing in this study, including: adverse event reporting, clinical evaluations including lesion size and clinical evaluation.

### Study Design

Patients showing lesions received an intravitreal injection of a peptide having an acetate salt of the structure of Formula III (ONL1204). Baseline best lesion size (area in square millimeters) was measured prior to treatment with ONL1204. Lesion size was then measured 24-weeks post treatment with ONL1204. The following doses were evaluated: 50 ug, 100 ug, and 200 ug (micrograms).

### Outcomes

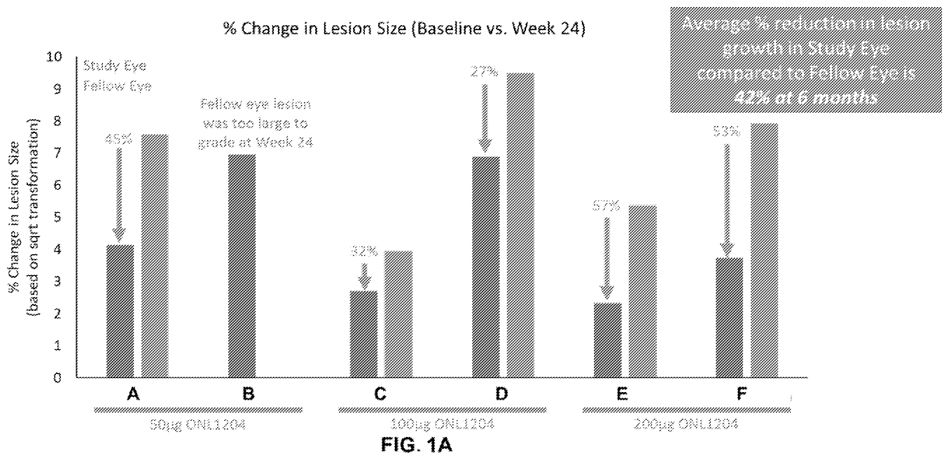

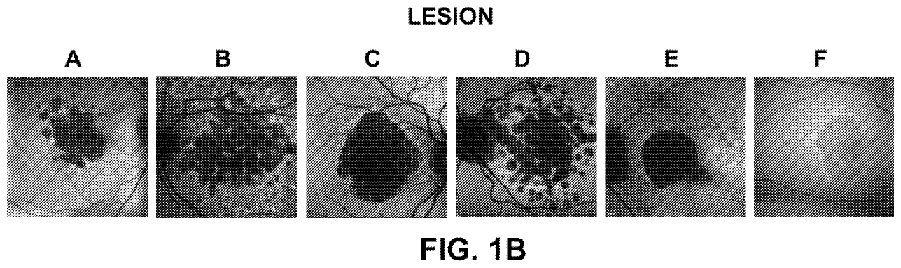

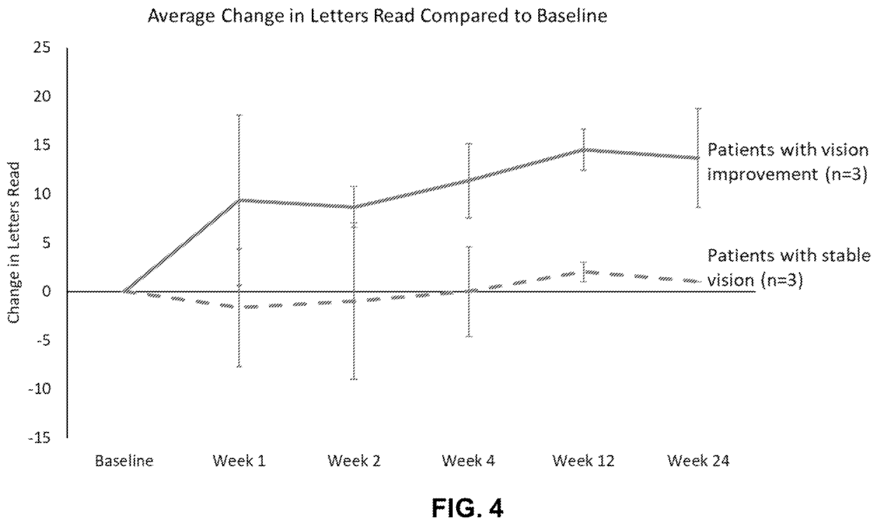

All patients receiving doses comprising 50 micrograms (ug), 100 ug, and 200 ug of ONL1204 showed a reduction in lesion size growth (e.g., compared to an untreated fellow eye). FIG. 1A shows study data for reduced lesion growth. An improvement in reducing lesion growth was observed for across patients receiving Fas Inhibitor ONL1204, as compared to the untreated fellow. FIG. 1B lesions corresponding to data shown in FIG. 1B. Table 1A and 1B show reduced lesion sizes associated with treated eyes at week 24. Percent (%) change is measured relative the untreated fellow eye. Table 2 shows improved visual function (e.g., BCVA as measured by letters read) in eyes having a patchy lesion when treated with ONL1204. FIG. 3A shows examples of patchy lesions and FIG. 3B shows examples of non-patchy or single lesions. FIG. 4 shows the average change in letters read as compared to baseline in eyes having patchy lesions (solid line) and single or non-patchy lesions (dotted line). Table 3A, 3B and 3C show reduced lesion sizes associated with treated eyes (Tables 3A and 3C) in addition to associated historical lesion growth (Tables 3A and 3B). For historical studies, historical data includes 24 weeks, from visit 1 (Vi) to visit 3 (V3), and the treatment phase data includes 24 weeks after first treatment, from visit 3 (V3) to visit 9 (V9), with patients receiving 2 injections of Fas Inhibitor ONL1204, 3 months apart. For “% Change Based on Sqrt Transformation” and similar measures, the square root of the lesion area was determined and the % difference between the starting value and the last timepoint was calculated. Percent (%) change is measured relative the untreated fellow eye.

TABLE 1A

| | | | Baseline | Week 24 | % Change |
|---|---|---|---|---|---|
| | | ONL1204 | Lesion | Lesion Size | Based on Sqrt |
| Patient | Eye | (ug) | Size (mm²) | (mm2) | Transformation |
| A | Study | 50 | 10.06 | 10.91 | 4.14% |
| A | Fellow | 0 | 7.12 | 8.24 | 7.58% |
| B | Study | 50 | 23.71 | 27.12 | 6.95% |
| B | Fellow | 0 | 39.95 | Too large | N/A |
| | | | | to grade | |
| C | Study | 100 | 20.1 | 21.2 | 2.70% |
| C | Fellow | 0 | 18.84 | 20.36 | 3.96% |
| D | Study | 100 | 24.16 | 27.61 | 6.90% |
| D | Fellow | 0 | 17.81 | 21.35 | 9.49% |
| E | Study | 200 | 9.37 | 9.81 | 2.32% |
| E | Fellow | 0 | 7.35 | 8.16 | 5.37% |
| F | Study | 200 | 9.09 | 9.78 | 3.73% |
| F | Fellow | 0 | 3.95 | 4.6 | 7.91% |

TABLE 1B

| | | ONL1204 | % Change in |
|---|---|---|---|
| Patient | Eye | (ug) | Lesion Growth |
| A | Study | 50 | −45% |
| A | Fellow | 0 | |
| B | Study | 50 | — |
| B | Fellow | 0 | |
| C | Study | 100 | −32% |
| C | Fellow | 0 | |
| D | Study | 100 | −27% |
| D | Fellow | 0 | |
| E | Study | 200 | −57% |
| E | Fellow | 0 | |
| F | Study | 200 | −53% |
| F | Fellow | 0 | |

TABLE 2

| | | | ETDRS Vision (Letters Read) | | | | | |
|---|---|---|---|---|---|---|---|---|
| Dose | | | Screening/ | Week | Week | Week | Week | Week |
| (μg) | Patient | Eye | Baseline | 1 | 2 | 4 | 12 | 24 |
| 50 | A | Study | 23 | 42 | 34 | 36 | 36 | 42 |
| | | Fellow | 77 | 74 | 70 | 75 | 77 | 76 |
| | B | Study | 26 | 33 | 33 | 33 | N/A | 35 |
| | | Fellow | 64 | 64 | 65 | 60 | N/A | 62 |
| 100 | C | Study | 14 | 13 | 13 | 10 | 15 | 15 |
| | | Fellow | 62 | 59 | 64 | 59 | 63 | 69 |
| | D | Study | 11 | 13 | 19 | 25 | 27 | 24 |
| | | Fellow | 43 | 38 | 43 | 50 | 50 | 53 |
| 200 | E | Study | 19/25* | 29 | 32 | 30 | 28 | 26 |
| | | Fellow | 78/84* | 91 | 87 | 85 | 83 | 76 |
| | F | Study | 39 | 31 | 30 | 38 | 41 | 40 |
| | | Fellow | 71 | 74 | 77 | 75 | 76 | 75 |

*Screening and Baseline Visits were on different days

TABLE 3A

| | | | V1 Lesion | V3 Lesion | V9 Lesion |
|---|---|---|---|---|---|
| | | ONL1204 | Size | Size | Size |
| Patient | Eye | (ug) | (mm²) | (mm²) | (mm²) |
| G | Study | 50 | 12.08 | 13.4 | 14.42 |
| G | Fellow | 0 | 10.12 | 11.1 | 12.22 |
| H | Study | 50 | 18.41 | 18.92 | 18.94 |
| B | Fellow | 0 | 18.75 | 19.76 | 19.94 |
| I | Study | 100 | 5.64 | 5.98 | 6.39 |
| I | Fellow | 0 | 1.14 | 1.22 | 1.37 |
| J | Study | 100 | 7.7 | 8.2 | 8.43 |
| J | Fellow | 0 | 10.31 | 10.6 | 10.96 |
| L | Study | Sham | 16.67 | 17.91 | 18.59 |
| L | Fellow | 0 | 13.12 | 14.99 | 15.28 |

TABLE 3B

| | | | % Change Nat His | % Difference in Lesion |
|---|---|---|---|---|
| | | ONL1204 | Phase (Using Sqrt | Growth Nat His Phase |
| Patient | Eye | (ug) | Transformation) | (Study vs Fellow Eye) |
| G | Study | 50 | 5.31% | 13% |
| G | Fellow | 0 | 4.73% | |
| H | Study | 50 | 1.38% | −88% |
| B | Fellow | 0 | 2.66% | |
| I | Study | 100 | 2.97% | −44% |
| I | Fellow | 0 | 3.45% | |
| J | Study | 100 | 3.20% | −17% |
| J | Fellow | 0 | 1.40% | |
| L | Study | Sham | 3.65% | 95% |
| L | Fellow | 0 | 6.89% | |

TABLE 3C

| | | | % Change Tx | % Difference in Lesion |
|---|---|---|---|---|
| | | ONL1204 | Phase (Using Sqrt | Growth Tx Phase |
| Patient | Eye | (ug) | Transformation) | (Study vs Fellow Eye) |
| G | Study | 50 | 3.74% | −24% |
| G | Fellow | 0 | 4.92% | |
| H | Study | 50 | 0.05% | −88% |
| B | Fellow | 0 | 0.45% | |
| I | Study | 100 | 3.37% | −44% |
| I | Fellow | 0 | 5.97% | |
| J | Study | 100 | 1.39% | −17% |
| J | Fellow | 0 | 1.68% | |
| L | Study | Sham | 1.88% | 95% |
| L | Fellow | 0 | 0.96% | |

### Example 2—Pharmacokinetics Studies in Rabbit Eye

### Objective

A study was performed to evaluate the ocular tissue concentration of a peptide having an acetate salt of the structure of Formula III (ONL1204) up to 168 days following a single intravitreal injection to both eyes and a repeat intravitreal injection on Day 90 in a select group. A total of 33 rabbits were evaluated on study to evaluate ONL1204 over 168 days.

### Study Design

Animals were observed for any abnormal observations prior to dosing, approximately weekly (for surviving animals through Week 12), and at euthanasia. Body weights were measured and recorded prior to first dose, approximately weekly (for surviving animals through Week 12), and prior to euthanasia. No ONL1204-related effects on body weights or clinical observations were evident during the study. Ophthalmic examinations were performed using the Hackett-McDonald Scoring System on Days 0, 7, 14, 28, 90, 97, 151, and 168. Pharmacokinetic analyses were performed on the vitreous humor concentration data.

Animals received a single dose of the test article, ONL1204, at 2 mg/mL (0.1 mg/eye once on Day 1 via intravitreal (IVT) injection). Seven animals received a repeat IVT dose at 2 mg/mL (0.1 mg/eye) on Day 90. The formulation contained the 2 mg/mL ONL1204 in Vehicle (4.5% mannitol/0.4% poloxamer-407, 10 mM acetic acid pH 4.5).

Vitreous humor, retina, and choroid were collected on days 1, 7, 14, 28, 42, 61, 90, 97, 151, and 168. Eyes were enucleated, snap frozen using liquid nitrogen, and kept on dry ice or frozen at −80° C. until dissected for collection of aqueous humor, vitreous humor, retina, choroid, iris-ciliary body (ICB), and lens.

### Sample Preparation

To homogenize ocular tissue samples, weighed amounts of control bovine vitreous humor, retina, and choroid were homogenized in USA scientific impact resistant microtubes containing 2.8 mm ceramic beads. Unknown rabbit vitreous humor, retina, and choroid samples were homogenized in USA scientific impact resistant microtubes containing 2.8 mm ceramic beads. Each vitreous humor sample was homogenized after the addition of 5000 microliters (uL) diluent (acetonitrile:water: 1 M hydrochloric acid (70:20:10, v/v/v). Retina tissues were homogenized after the addition of diluent (IPA:NH4OH (1000:1, v/v)) at a 1:5 ratio (parts tissue to parts diluent), and choroid samples were homogenized following addition of diluent at a 1:19 ratio. Tissues were homogenized at 5500 rpm for 3×30 second cycles with 20 second pauses between cycles until homogenized. Choroid went through two runs of homogenization, and retina and vitreous humor samples went through one run of homogenization.

Preparation of Calibration Stock and Working Standards: A stock calibration standard was prepared in dimethylsulfoxide (DMSO) at a concentration of 500 ug/mL for ONL1204. Working calibration standards were prepared for vitreous humor by serial dilution of working stock solution with acetonitrile:water:formic acid (25:75:0.1, v/v/v) over a range of 500 ng/mL to 500,000 ng/mL ONL1204. Working calibration standards were prepared for choroid and retina by serial dilution of working stock solution with acetonitrile:water:formic acid (25:75:0.1, v/v/v) over a range of 5.00 ng/mL to 1,000 ng/mL ONL1204.

Preparation of Standards, Unknowns, Blanks, and Blanks with Internal Standard for Vitreous Humor Analysis: In a polypropylene tube, 10 uL of working calibration standard was added to 90 uL control blank vitreous humor. For blanks and blanks with internal standard, 100 uL of control blank bovine vitreous humor was added. Five hundred (500) uL of acetonitrile:water:1M hydrochloric acid (70:20:10, v/v/v) was added to each standard and 500 uL of acetonitrile:water:1M hydrochloric acid (70:20:10, v/v/v) was added to blanks. Standards and blanks were vortex mixed. One hundred (100) uL of each standard, blank or unknown vitreous humor sample homogenate was then aliquoted. One hundred (100) uL of IPA:NH4OH (1000:1, v/v) was added to each sample. The samples were vortex mixed for 1 minute, then centrifuged for 10 minutes at 4,000 rpm (4° C.). In an autosampler plate, 50.0 uL of working internal standard (WIS) (5,000 ng/mL APi1887 in acetonitrile:water:formic acid (25:75:0.1, v/v/v)) was added to 50.0 uL supernatant. Fifty (50.0) uL of acetonitrile:water: 1M hydrochloric acid (70:20:10, v/v/v) was added to blank without IS. Two hundred (200) uL of water was added. The samples were then mixed using a multichannel pipette, and supernatant transferred to an autosampler plate for analysis.

Preparation of Standards, Unknowns, Blanks, and Blanks with Internal Standard for Retina and Choroid: In a 96-well plate, 100 uL of control retina or choroid homogenate was added for calibration samples and unknowns. For blanks and blanks with internal standard, 100 uL of control bovine retina or choroid tissue homogenate was added. Twenty (20) uL of WIS (5,000 ng/mL APi1887 in acetonitrile:water:formic acid [25:75:0.1, v/v/v]) was added to each calibration standard and blank with IS sample. Twenty (20) uL acetonitrile:water:formic acid (25:75:0.1, v/v/v) was added to each blank without IS sample. Three-hundred (300) uL of IPA:NH4OH (1000:1, v/v) was added to each choroid or retina sample. The samples were then vortex mixed for 5 minutes. The samples were allowed to set for 15 minutes, vortex mixed for an additional minute, and centrifuged for 10 minutes at 4,000 rpm (4° C.).

Three-hundred-fifty (350) uL of each sample was then aliquoted to a 96-well plate. The samples were dried with nitrogen at 35° C. Two-hundred (200) uL of acetonitrile:water:formic acid (25:75:0.1, v/v/v) was added to reconstitute each sample. The samples were then vortex mixed for 4 minutes, and transferred to a 96-well autosampler plate for analysis.

Based on the results, the t½ for animals receiving a single dose was 279.8 days (0.1 mg/eye, days 0 to 168). Table 3 shows drug concentrations in the vitreous humor and retinal issue. ONL1204 was detected in the vitreous humor across all time points analyzed. Retinal tissue is generally difficult to assay, wherein detection in the retina represents the minimal amount of ONL1204 present (i.e., the values do not likely overestimate the concentration of ONL1204 in retinal tissue (limit of quantification (LLOQ)=0.500 ng/mL, 3.00 ng/g for the retina, unless marked with “<LLOQ*” which indicates LLOQ=1.00 ng/mL, 6.0 ng/g; ** denotes animal group receiving a second injection at day 90). Nonetheless, ONL1204 was detected in retinal tissue as well as the vitreous humor after 168 days.

TABLE 3

| | | Conc. of ONL1204 | |
|---|---|---|---|
| Sample | | Vitreous | Retina |
| ID** | Day | Humor (ug) | (ng/g) |
| 1A-Right | 1 | 76 | <LLOQ |
| 1A-Left | 1 | 48.3 | 12.7 |
| 1B-Right | 1 | 80.9 | <LLOQ |
| 1B-Left | 1 | 89.6 | <LLOQ |
| 1C-Right | 1 | 74.9 | <LLOQ |
| 1C-Left | 1 | 77.1 | <LLOQ |
| 2A-Right | 7 | 83 | <LLOQ |
| 2A-Left | 7 | 41.6 | <LLOQ |
| 2B-Right | 7 | 82.6 | <LLOQ |
| 2B-Left | 7 | 83.6 | <LLOQ |
| 2C-Right | 7 | 79.2 | <LLOQ |
| 2C-Left | 7 | 67.8 | 85.2 |
| 3A-Right | 14 | 67.3 | <LLOQ |
| 3A-Left | 14 | 67.4 | <LLOQ |
| 3B-Right | 14 | 54.2 | <LLOQ |
| 3B-Left | 14 | 70.9 | <LLOQ |
| 3C-Right | 14 | 63.2 | 5.95 |
| 3C-Left | 14 | 74.5 | <LLOQ |
| 4A-Right | 28 | 76.9 | <LLOQ |
| 4A-Left | 28 | 70.2 | <LLOQ |
| 4B-Right | 28 | 74.4 | <LLOQ |
| 4B-Left | 28 | 82 | <LLOQ |
| 4C-Right | 28 | 69.9 | <LLOQ |
| 4C-Left | 28 | 63.9 | <LLOQ |
| 5A-Right | 42 | 71.7 | <LLOQ |
| 5A-Left | 42 | 69.3 | <LLOQ |
| 5B-Right | 42 | 65.3 | <LLOQ |
| 5B-Left | 42 | 75.7 | <LLOQ |
| 5C-Right | 42 | 60.9 | <LLOQ |
| 5C-Left | 42 | 62.3 | <LLOQ |
| 6A-Right | 61 | 54.8 | <LLOQ |
| 6A-Left | 61 | 55.2 | <LLOQ |
| 6B-Right | 61 | 58.3 | <LLOQ |
| 6B-Left | 61 | 54.5 | <LLOQ |
| 6C-Right | 61 | 59.9 | <LLOQ |
| 7A-Right | 90 | 63.7 | <LLOQ* |
| 7A-Left | 90 | 61.5 | <LLOQ* |
| 8A-Right | 90 | 54.1 | <LLOQ* |
| 8A-Left | 90 | 51 | <LLOQ* |
| 7B-Right** | 97 | 13 | 9.55 |
| 7B-Left | 97 | 135 | <LLOQ* |
| 8B-Right** | 97 | 127 | 8.52 |
| 8B-Left | 97 | 113 | <LLOO* |
| 8C-Right | 151 | 99.1 | <LLOQ* |
| 8C-Left** | 151 | 103 | 15.6 |
| 9A-Right** | 151 | 113 | 52.9 |
| 9A-Left | 151 | 124 | 9.54 |
| 9B-Right | 168 | 99.8 | 83.9 |
| 9B-Left | 168 | 103 | <LLOQ* |
| 9C-Right | 168 | 75.9 | <LLOQ* |
| 9C-Left | 168 | 85.6 | <LLOQ* |
| 10B-Right | 168 | 48.7 | 32.9 |
| 10B-Left | 168 | 45.7 | 8.4 |
| 10C-Right | 168 | 55.8 | <LLOQ* |
| 10C-Left | 168 | 44.7 | <LLOQ* |

Right or left designates the right or left eye, respectively.

### Example 3—Sodium Iodate (NaIO₃) Challenge Study

### Objective

The objective of this study was to test the duration of activity (e.g., protection) of ONL1204 in the male rabbit NaIO₃ model by treating animals with test article (ONL1204) on Day 0 and staggering the day of NaIO₃ insult to assess duration of protection offered by ONL1204. Demonstrating a protective effect of ONL1204 at extended timepoints would support the use of ONL1204 ophthalmic solution in chronic ophthalmic diseases.

### Study Design

Test Article: Test Article: ONL 1204 Formulation; Administration Route: 50 ul by IVT injection; Frequency of Administration: Single dose on Day 0; Formulations: Group 1-4; 9: 2 mg/ml ONL 1204 in 4.5% mannitol, 0.4% poloxamer 407, 10 mM acetate buffer pH 4.5, Group 5-8: 0.5 mg/ml ONL 1204 in 4.5% mannitol, 0.4% poloxamer 407, 10 mM acetate pH 4.5; Dose: Group 1-4; 9: 100 μg per eye, Group 5-8: 25 μg per eye

NaIO₃ solution: Challenge Article: NaIO₃; Administration Route: IV; Frequency of Administration: Day 4 (day 7 endpoint), 14 (day 17 endpoint), 28 (day 31 endpoint), 59 (day 61 endpoint), or 78 (day 81 endpoint (rechallenged group)). Solution: 20 mg/ml in 0.9% Injectable Saline; Dose: 17 or 18 mg/kg body weight

Test System: Species: Rabbit; Expected Age: Animals were between 3 and months of age; Expected Body Range: Approximately 2.0 Kg, Identification Animals were individually housed and identified via cage card and ear ID and/or ear tattoo.

Intravitreal Injection: On Day 0, eyes were anesthetized with proparacaine and pupils dilated with tropicamide. Animals were anesthetized with isoflurane vapors to effect. A few drops of Betadine were placed on the eye and then rinsed with ophthalmic eye wash. A clean speculum was placed in the eye (rinsed in Betadine and sterile saline) and the temporal sclera was marked with calipers 3.5 mm from the corneal border. A BD Ultra-Fine 31 g, 3/10 cc, 5/16 in, Insulin Syringe with 50 ul of test article of 0.5 mg/ml or 2.0 mg/ml ONL1204 was inserted though indentation into the mid-vitreous approximately 620 from the horizontal axis (28° from the vertical axis) and test article was injected into the vitreous. In order to reduce the potential for reflux of the test article the needle remained in the vitreous for at least 10 seconds following injection. No reflux was noted on the dosing record.

Model Induction: A 20 mg/ml solution of NaIO₃ was prepared in 0.9% injectable saline aliquoted into 4 batches and frozen at −20° C. For Groups 1 to 8, 1 batch of NaIO₃ was thawed prior to injection. Groups 9-10 were added after the Study was underway therefore a new solution of 20 mg/ml NaIO₃ was prepared in 0.9% injectable saline and frozen at −20° C. and thawed prior to injection. Test article and NaIO₃ were injected into the left ear.

Animals were weighed and anesthetized with isoflurane vapors to effect. The left ear was shaved and swabbed with 10% alcohol prior to injection. An indwelling catheter was placed in the left marginal ear vein and flushed with 0.9% injectable saline. Animals were weighed just prior to dosing and Groups 1 to 8 received 18 mg NaIO₃/kg bodyweight followed by a flush of 0.3 ml 0.9% injectable saline. Groups 9-10 received 16 mg NaIO₃/kg bodyweight followed by a flush of 0.3 ml 0.9% injectable saline. All injections were performed without complication and no reflux or leakage was noted.

Fluorescein Angiography: Eyes were anesthetized with proparacaine and pupils dilated with tropicamide and phenylephrine. Animals were anesthetized with isoflurane vapors to effect. 50ul of 25% sodium fluorescein was injected into the marginal ear vein and allowed to circulate for at least 2 minutes prior to imaging. In animals with intact retinal pigment epithelial cell pigmentation, it is not possible to stimulate or visualize the light emitted by fluorescein circulating in the choroidal vasculature. NaIO₃ causes one or more regions of atrophy and depigmentation which creates a window into the choroid. Images were captured to best map the full extent of visible choroid/atorphy in both eyes.

Data Analysis: The area of RPE depigmentation is reported as a unitless value provided by the ImageJ software. In certain cases where the damage extended well into the periphery it was not possible to image the entire edge of the affected area. In some cases, a typical lesion was not created in either eye. In these cases, it is possible to measure the accumulation of lipofuscin, the phagocytized shed outer segments normally processed by the RPE, which builds up in unhealthy RPE. In these cases, ImageJ was used to highlight the autofluorescence around the Optic Nerve Head by thresholding the image with a 100/20 window level. The resulting area was then measured.

### Results

The mean lesion size in both eyes of 3 male rabbits, N=6, were used as negative control, against which the protective effect of ONL1204 treatment was compared. On Day 4, there were equally small lesions in both treatment groups and no statistical difference in lesion area. Both Groups were significantly lower than negative control. On Day 14, animals treated with 100 ug had significantly smaller lesion size then both negative controls and those treated with 25 ug. On Day 28, animals treated with 100 ug had significantly smaller lesion size then both negative control animals and those treated with 25 ug. A substantive reduction in mean lesion size was observed across challenge time points. On Day 59, the mean lesion size for animals treated with 25 ug and 100 ug was 40% and 50% smaller respectively compared to negative controls. Results are shown in FIG. 1.

### Example 4—Pharmacokinetics Studies in Minipig

### Objective

A study was performed in minipig to evaluate the ocular tissue concentrations of a peptide having the structure of Formula III (ONL1204) following a single intravitreal injection.

### Study Design

Animals received a single dose of the test article, ONL1204, at 2 mg/mL (0.1 mg/eye once on Day 1 via intravitreal (IVT) injection). Seven animals received a repeat IVT dose at 2 mg/mL (0.1 mg/eye) on Day 90. The formulation contained the 2 mg/mL ONL1204 in Vehicle (4.5% mannitol/0.4% poloxamer-407, 10 mM acetic acid pH 4.5). Vitreous humor, retina, and choroid were collected on days 1, 7, 14, 28, and 88.

### Pharmacokinetics

Table 4 shows drug concentration results in the vitreous humor, retinal issue, and choroid. ONL1204 was detected in the vitreous humor across all time points analyzed. Due to assay limitations, detection in the retina represents the minimal amount of ONL1204 present (i.e., the values do not likely overestimate the concentration of ONL1204 in retinal tissue) (limit of quantification (LLOQ) for vitreous humor=0.5 ug/mL; LLOQ for retina=0.500 ng/mL, 2.5 ng/g unless noted by “<LLOQ*” indicating LLOQ=1.00 ng/mL, 5.0 ng/g; LLOQ for choroid=1.00 ng/mL, 20.0 ng/g).

TABLE 4

| | | | Concentration | | |
|---|---|---|---|---|---|
| Animal | Dose | | Vitreous | Retina | Choroid |
| ID | (ug/eye) | Day | humor (μg) | (ng/g) | (ng/g) |
| 1 | Vehicle | 6 | <LLOQ | <LLOQ | <LLOQ |
| 2 | | 6 | <LLOQ | <LLOQ | <LLOQ |
| 3 | | 6 | <LLOQ | <LLOQ | <LLOQ |
| 4 | | 28 | <LLOQ | <LLOQ | <LLOQ |
| 5 | | 28 | <LLOQ | <LLOQ | <LLOQ |
| 6 | | 28 | <LLOQ | <LLOQ | <LLOQ |
| 7 | | 88 | <LLOQ | <LLOQ* | <LLOQ |
| 8 | | 88 | <LLOQ | <LLOQ* | <LLOQ |
| 9 | | 88 | <LLOQ | <LLOQ* | <LLOQ |
| 10 | 200 | 6 | 130 | <LLOQ | 32.4 |
| 11 | | 6 | 106 | 4.16 | 352 |
| 12 | | 6 | 111 | 49.3 | 182 |
| 13 | | 28 | 156 | 6.14 | 166 |
| 14 | | 28 | 133 | <LLOQ | <LLOQ |
| 15 | | 28 | 107 | <LLOQ | 33.5 |
| 16 | | 88 | 95.1 | <LLOQ* | 31 |
| 17 | | 88 | 75.5 | <LLOQ* | 62.5 |
| 18 | | 88 | 54.0 | <LLOQ* | 21 |
| 19 | 300 | 6 | 205 | 9.5 | <LLOQ |
| 20 | | 6 | 141 | 12.1 | 125 |
| 21 | | 6 | 153 | <LLOQ | 178 |
| 22 | | 28 | 124 | 39.3 | 77.7 |
| 23 | | 28 | 232 | 25.5 | 119 |
| 24 | | 28 | 184 | 6.59 | 50.5 |
| 25 | | 88 | 88.3 | <LLOQ* | <LLOQ |
| 26 | | 88 | 137 | <LLOQ* | 89.4 |
| 27 | | 88 | 99.6 | <LLOQ* | <LLOQ |

While preferred embodiments of the present disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein may be employed in practicing the invention. It is intended that the following claims define the scope of the embodiments disclosed herein, and that methods and structures within the scope of these claims and their equivalents be covered thereby.

### SEQUENCES

| SEQ ID NO | SEQUENCE |
|---|---|
| 1 | HHIYLGAVNYIY |
| 2 | YIYNVAGLYIHH |

1. A method of improving visual function in an eye of an individual having a patchy ocular lesion, wherein the method comprises:
administering a peptide to the eye, wherein the peptide comprises an amino acid sequence HHIYLGAVNYIY or variant sequence thereof, or a pharmaceutically acceptable salt thereof.

2. A method of treating an ocular lesion in an eye of an individual, the method comprising:
administering a peptide to the eye, wherein the peptide comprises an amino acid sequence HHIYLGAVNYIY or variant sequence thereof, or a pharmaceutically acceptable salt thereof.

3. A method of improving visual function in an eye of an individual having a patchy ocular lesion, wherein the method comprises:
(a) administering a first composition to the eye, wherein the first composition comprises:
a peptide comprising an amino acid sequence HHIYLGAVNYIY or variant sequence thereof, or a pharmaceutically acceptable salt thereof; and
(b) administering a second composition to the eye, wherein the second composition comprises the peptide, and wherein the second composition is administered about 10 weeks or greater after administering the first composition.

4. A method of treating an ocular lesion in an eye of an individual, the method comprising:
(a) administering a first composition to the eye, wherein the first composition comprises:
a peptide comprising an amino acid sequence HHIYLGAVNYIY or variant sequence thereof, or a pharmaceutically acceptable salt thereof; and
(b) administering a second composition to the eye, wherein the second composition comprises the peptide, and wherein the second composition is administered about 10 weeks or greater after administering the first composition.

5. The method of any one of claims 3-4, wherein the second composition is administered about 12 weeks after administering the first composition.

6. The method of any one of claims 1-5, wherein administering the peptide reduces the rate of growth (e.g., an increase in total cross-sectional area) of the patchy ocular lesion.

7. The method of any one of claims 1-6, wherein the patchy ocular lesion has a total size (e.g., cross-sectional area) of about 3 mm² or greater.

8. The method of any one of claims 1-7, wherein the patchy ocular lesion has a total size (e.g., cross-sectional area) of about 3 mm² to 20 mm².

9. The method of any one of claims 1-8, wherein the patchy ocular lesion comprises a retinal lesion.

10. The method of any one of claims 1-9, wherein the peptide or the pharmaceutically acceptable salt thereof has a half-life in the vitreous humor of (e.g., at least) more than about 30 days.

11. The method of claim 10, wherein the half-life in the vitreous humor (e.g., at least) is more than about 90 days.

12. The method of claim 10, wherein the half-life in the vitreous humor (e.g., at least) is more than about 180 days.

13. The method of claim 10, wherein the half-life in the vitreous humor (e.g., at least) is more than about 90 days to about 300 days.

14. The method of any one of claims 1-13, wherein the method comprises using the vitreous humor as a depot to provide the peptide or the pharmaceutically acceptable salt thereof to retinal tissue in the eye, wherein the peptide or the pharmaceutically acceptable salt thereof is present in the vitreous humor more than about 30 days after administration.

15. The method of claim 14, the peptide or the pharmaceutically acceptable salt thereof is present in the vitreous humor more than about 90 days after administration.

16. The method of claim 14, the peptide or the pharmaceutically acceptable salt thereof is present in the vitreous humor more than about 180 days after administration.

17. The method of any one of claims 1-16, wherein the peptide or the pharmaceutically acceptable salt thereof is provided to the retina from the vitreous humor for more than about 30 after administration.

18. The method of claim 17, wherein the peptide or the pharmaceutically acceptable salt thereof is provided to the retina from the vitreous humor for more than about 90 after administration.

19. The method of claim 17, wherein the peptide or the pharmaceutically acceptable salt thereof is provided to the retina from the vitreous humor for more than about 180 after administration.

20. The method of any one of claims 1-19, wherein the variant sequence comprises an amino acid substitution.

21. The method of claim 20, wherein the variant sequence comprises one amino acid substitution.

22. The method of any one of claims 1-21, wherein the peptide further comprises a modification.

23. The method of claim 22, wherein the modification comprises a modified amino acid.

24. The method of any one of claims 1-23, wherein the peptide comprises an amidated C-terminus.

25. The method of any one of claims 1-24, wherein the peptide has the structure of Formula I or a pharmaceutically acceptable salt thereof.

26. The method of any one of claims 1-25, wherein the peptide has the structure of Formula III: 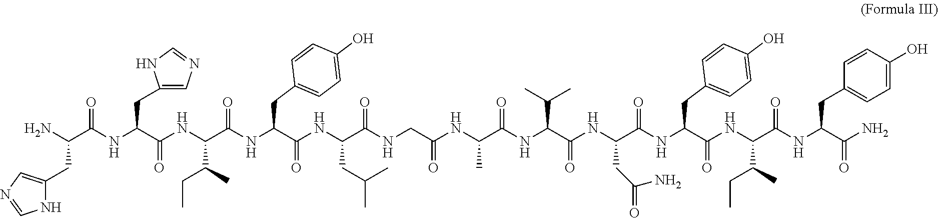 [SMILES: CC[C@H](C)[C@H](NC(=O)[C@H](Cc1ccc(O)cc1)NC(=O)[C@H](CC(N)=O)NC(=O)[C@@H](NC(=O)[C@H](C)NC(=O)CNC(=O)[C@H](CC(C)C)NC(=O)[C@H](Cc1ccc(O)cc1)NC(=O)[C@@H](NC(=O)[C@H](Cc1cnc[nH]1)NC(=O)[C@@H](N)Cc1cnc[nH]1)[C@@H](C)CC)C(C)C)C(=O)N[C@@H](Cc1ccc(O)cc1)C(N)=O]
or a pharmaceutically acceptable salt thereof.

27. The method of any one of claims 1-26, wherein the method comprises administering the pharmaceutically acceptable salt thereof.

28. The method of claim 27, wherein the pharmaceutically acceptable salt is an acetate salt.

29. The method of claim 28, wherein the pharmaceutically acceptable salt is a polyacetate salt.

30. The method of claim 29, wherein the polyacetate salt is a triacetate salt.

31. The method of claim 27, wherein the pharmaceutically acceptable salt is a hydrochloride salt.

32. The method of any one of claims 1-31, wherein the method comprises administering a composition comprising the peptide.

33. The method of any one of claim 32, wherein the composition (e.g., each composition or the first/second composition) further comprises one or more excipients.

34. The method of any one of claims 32-33, wherein the composition (e.g., each composition or the first/second composition) further comprises a surfactant.

35. The method of claim 34, wherein the surfactant is a non-ionic surfactant.

36. The method of claim 35, wherein the surfactant is a polysorbate, a polyethoxylated castor oil derivative, a polyethoxylated fatty acid, a polyethoxylated alcohol, a polyoxyethylene-polyoxypropylene block copolymer, or an oxyethylated tertiary octylphenol formaldehyde polymer.

37. The method any one of claims 34-36, wherein the surfactant forms about 0.01% to about 20% weight/weight of the composition.

38. The method of claim 37, wherein the surfactant forms about 0.05% to about 10% weight/weight of the composition.

39. The method of any one of claims 32-38, wherein the composition (e.g., each composition or the first/second composition) further comprises a tonicity adjusting agent, a buffering agent, or a combination thereof.

40. The method of any one of claims 32-39, wherein the composition is buffered at a pH of 2.5 to 7.5.

41. The method of any one of claims 32-40, wherein the composition comprises 50 micrograms (ug) to 500 ug of the peptide.


SEQUENCE LISTING (6 sequences)

SEQ ID NO: 1 - protein, 12 aa, synthetic construct
HHIYLGAVNYIY

SEQ ID NO: 2 - protein, 12 aa, synthetic construct
YIYNVAGLYIHH

SEQ ID NO: 3 - protein, 12 aa, synthetic construct
  SITE 3: note = Any amino acid with a 1-propyl, 2-propyl, 2-methyl-prop-2-yl, C3-6-cycloalkyl, C4-6-cycloalkenyl, tetrahydrofuran-2-yl, tetrahydrofuran-3-yl, tetrahydrothienyl-2-yl, tetrahydrothienyl-3-yl, tetrahydropyran-2-yl, tetrahydropyran-3-yl, tetrahydropyran- 4-yltetrahydrothiopyran-2-yl, tetrahydrothiopyran-3-yl, tetrahydrothiopyran-4-yl or 1-CH(OR5)CH3 side chain
  SITE 4: note = Tyrosine or modified tyrosine
  SITE 10: note = Tyrosine or modified tyrosine
  SITE 11: note = Any amino acid with a 1-propyl, 2-propyl, 2-methyl-prop-2-yl, C3-6-cycloalkyl, C4-6-cycloalkenyl, tetrahydrofuran-2-yl, tetrahydrofuran-3-yl, tetrahydrothienyl-2-yl, tetrahydrothienyl-3-yl, tetrahydropyran-2-yl, tetrahydropyran-3-yl, tetrahydropyran- 4-yltetrahydrothiopyran-2-yl, tetrahydrothiopyran-3-yl, tetrahydrothiopyran-4-yl or 1-CH(OR5)CH3 side chain
  SITE 12: note = Tyrosine or modified tyrosine
HHXXLGAVNXXX

SEQ ID NO: 4 - protein, 12 aa, synthetic construct
  SITE 1: note = Tyrosine or modified tyrosine
  SITE 2: note = Any amino acid with a 1-propyl, 2-propyl, 2-methyl-prop-2-yl, C3-6-cycloalkyl, C4-6-cycloalkenyl, tetrahydrofuran-2-yl, tetrahydrofuran-3-yl, tetrahydrothienyl-2-yl, tetrahydrothienyl-3-yl, tetrahydropyran-2-yl, tetrahydropyran-3-yl, tetrahydropyran- 4-yltetrahydrothiopyran-2-yl, tetrahydrothiopyran-3-yl, tetrahydrothiopyran-4-yl or 1-CH(OR5)CH3 side chain
  SITE 3: note = Tyrosine or modified tyrosine
  SITE 9: note = Tyrosine or modified tyrosine
  SITE 10: note = Any amino acid with a 1-propyl, 2-propyl, 2-methyl-prop-2-yl, C3-6-cycloalkyl, C4-6-cycloalkenyl, tetrahydrofuran-2-yl, tetrahydrofuran-3-yl, tetrahydrothienyl-2-yl, tetrahydrothienyl-3-yl, tetrahydropyran-2-yl, tetrahydropyran-3-yl, tetrahydropyran- 4-yltetrahydrothiopyran-2-yl, tetrahydrothiopyran-3-yl, tetrahydrothiopyran-4-yl or 1-CH(OR5)CH3 side chain
XXXNVAGLXXHH

SEQ ID NO: 5 - protein, 12 aa, synthetic construct
YVYNVAGLYVHH

SEQ ID NO: 6 - protein, 12 aa, synthetic construct
  SITE 10: note = Histidine with C-terminal-1-Acetylpiperazine
YVYNVAGLYVHH